(19) United States
(12) Patent Application Publication
(10) Pub. No.: US 2010/0041592 A1
(43) Pub. Date: Feb. 18, 2010

(54) Use of Amphiphilic Biocompatible Polymers for Solubilization of Hydrophobic Drugs

(76) Inventors: Alexander V. Kabanov, Omaha, NE (US); Robert Luxanhofer, Dresden (DE); Rainar Frank Jordan, Dresden (DE)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/492,660

(22) Filed: Jun. 26, 2009

Related U.S. Application Data
(60) Provisional application No. 61/133,154, filed on Jun. 26, 2008.
(60) Provisional application No. 61/134,209, filed on Jul. 8, 2008.

(51) Int. Cl. A61K 38/13; A61K 31/337; A61K 31/445; A61P 35/00

(52) U.S. Cl. 514/11; 514/449; 514/315

(57) ABSTRACT

The present invention provides polymer aggregates as delivery vehicles for therapeutics and diagnostics. The present invention additionally provides methods of synthesis and uses for such aggregates.

This application claims priority under 35 U.S.C. §119(e) to U.S. Provisional Patent Application No. 61/133,154, filed on Jun. 26, 2008 and U.S. Provisional Patent Application No. 61/134,209, filed on Jul. 8, 2008. The foregoing applications are incorporated by reference herein.

This invention was made with government support under Grant No. 2R01CA89225 awarded by the National Institutes of Health. The government has certain rights in the invention.

### FIELD OF THE INVENTION

The present invention relates generally to the solubilization of biologically active compounds with polymeric excipients of amphiphilic nature. The present invention relates to compositions and methods for the delivery of therapeutic and diagnostic agents, particularly hydrophobic compounds, to a patient.

### BACKGROUND OF THE INVENTION

A great number of potent drugs and potential drug candidates have a low solubility in water or aqueous solutions, thus limiting their scope of use. It is therefore necessary or beneficial to be able to solubilize or formulate hydrophobic drugs in aqueous media. The solubilized drugs may have improved dispersion in the aqueous media and/or increased stability in the aqueous dispersions.

Various methods to solubilize or disperse drugs have been developed. These methods are typically based on the use of solvents, surfactants, chelating agents or other drug delivery systems such as liposomes. These methods have one or more disadvantages related to the toxicity of the excipients, difficult formulation procedures, and/or limited stability of the formulations in aqueous media. Stability is a particularly problematic upon the dilution encountered when administered to a patient.

Copolymers comprising at least one hydrophilic and one hydrophobic block (amphiphilic block copolymers) have been shown to be effective for the solubilization of drugs of limited solubility in aqueous media.

U.S. Patent Application Publication No. 2004/0185101 discloses polymeric compositions with the capability to solubilize hydrophobic drugs in aqueous media. The biodegradable ABA-type or BAB-type block copolymers used in this approach can markedly increase the solubility of hydrophobic drugs, such as paclitaxel, in aqueous solution. However, one disadvantage of this approach is that the amount of polymer excipient is very high, typically between 10 and 30%. Moreover, the loading capacity of these compositions is very limited with a loading capacity of <10% (w/w) for paclitaxel and less than 1% (w/w) for cyclosporin A.

To date, few nontoxic biocompatible formulations are known for the solubilization of paclitaxel. The only formulation commercially available utilizes a 1/1 mixture of Cremophor EL® and dehydrated ethanol (v/v). While this formulation is able to solubilize relatively large amounts of paclitaxel (6 mg/ml) in the pure formulation which must then be diluted to obtain in administrable aqueous solution), it can also cause severe side effects in patients. It is therefore highly desirable to find new ways to formulate paclitaxel and other drugs in aqueous media suitable for intravenous injection to patients.

### SUMMARY OF THE INVENTION

In accordance with the instant invention, compositions and methods are provided for the solubilization of compounds, particularly hydrophobic compounds. In accordance with one aspect of the invention, compositions are provided comprising 1) at least one amphiphilic block copolymer comprising at least one hydrophilic segment and at least one hydrophobic segment, and 2) at least one hydrophobic compound, particularly a therapeutic agent. The composition may further comprise at least one pharmaceutically acceptable carrier. In a preferred embodiment, the hydrophilic segment is a hydrophilic poly(2-oxazoline) and the hydrophobic segment is a hydrophobic poly(2-oxazoline). In a particular embodiment, the hydrophilic segment is poly(2-methyl-2-oxazoline) or poly(2-ethyl-2-oxazoline) and the hydrophobic segment is poly(2-alkyl-2-oxazoline), wherein the alkyl comprises three to six carbons (e.g., butyl).

In accordance with another aspect of the instant invention, methods for delivering at least one compound to a subject are provided. The methods comprise administering at least one composition of the instant invention to a subject. In a particularly embodiment, the compound is a hydrophobic compound, particularly a therapeutic agent.

In accordance with yet another aspect of the instant invention, methods of treating a disorder or disease in a patient in need thereof are provided. The methods comprise administering at least one composition of the instant invention to the patient. In a particular embodiment, the disease is cancer and the administered compound is a chemotherapeutic agent such as a taxane.

### BRIEF DESCRIPTIONS OF THE DRAWING

FIG. 1A is a graph demonstrating the loading of paclitaxel in compositions comprising LXRB20 and increasing amounts of paclitaxel. FIG. 1B is a graph demonstrating the loading of paclitaxel in compositions comprising LXRB10, LXRB15, or LXRB20. The columns show the paclitaxel concentration in aqueous micelle solution as determined by HPLC. The line graph represents the loading efficiency ([paclitaxel]_{det}/[paclitaxel]₀×100%).

FIG. 2 is a graph depicting the amount of paclitaxel loaded with increasing amounts of LXRB15 and the loading efficiency.

FIGS. 3A-3C are graphs depicting the amount of paclitaxel loaded and the loading efficiency with various polymers.

FIGS. 4A and 4B are graphs depicting the toxicity of paclitaxel (Taxol®) solubilized in LXRB20 of paclitaxel solubilized in Cremophor EL®. FIG. 4C is a graph demonstrating the toxicity of paclitaxel (Taxol®) alone, paclitaxel (Taxol®) solubilized in LXRB10 (0.1% wt), or paclitaxel (Taxol®) solubilized in LXRB10 diluted.

FIGS. 5A-5D provide graphs showing the fluorescence intensity and I₁/I₃ ratios of pyrene solutions (5×10⁻⁷ M in PBS) at various concentrations of P1-P4, respectively, at 25° C.

FIG. 6A is a graph of the pyrene fluorescence spectra recorded at room temperature in aqueous solutions of 2-nonyl-2-oxazoline based block copolymer NOx₁₀-b-MeOx₃₂ (2.1×10⁻⁴ M), Pluronic® P85 (2.2×10⁻³ M), and the 2-butyl-2-oxazoline based MeOx₃₆-b-BuOx₃₀-b-MeOx₃₆ (P3, 7.1×10⁻⁴ M). FIG. 6B provides a comparison between pyrene fluorescence spectra in P3 (7.1×10⁻⁴ M) and an ionic liquid (1-butyl-2,3-dimethylimidazolium chloride) ([pyrene]=5×10⁻⁷ M, λ_{exc}=333 nm, pH 7.2).

FIGS. 7A and 7B provide a comparison of ¹H-NMR spectra of P4 (FIG. 7A) and P5 (FIG. 7B) (300K, 400 MHz, normalized for methyl or ethyl side chain, respectively) in deuterated chloroform (no aggregates present) and D₂O (formation of polymeric micelles). Signals 1-4 (CDCl₃) and 1′-4′ (D₂O) originated from butyl side chains in the hydrophobic block of P4 and P5, signals 5/5′ originated from polymer main chain, and signals 6/6′ and 7/7′ originated from side chains in the hydrophilic block.

FIGS. 8A-8D show the solubilization of paclitaxel (PTX) with amphiphilic poly(2-oxazoline) block copolymers using the film method. FIG. 8A shows the solubilization of paclitaxel with P2 (10 mg/mL) and the loading efficiency for paclitaxel concentrations of 4 mg/mL, 7 mg/mL, and 10 mg/mL. FIG. 8B shows the solubilization of paclitaxel using P1-P4 (10 mg/mL) and the loading efficiencies at a paclitaxel concentration of 4 mg/mL. FIG. 8C shows the solubilization of paclitaxel with P3 (2 mg/mL) and the loading efficiency for paclitaxel concentrations of 100 μg/mL, 500 μg/mL and 1 mg/mL. FIG. 8D shows the solubilization of paclitaxel using P1-P3 (2 mg/mL) and the loading efficiencies at a paclitaxel concentration of 500 μg/mL. Data is presented as means±SEM (n=3 except for FIG. 8C for 1 mg/mL paclitaxel where n=1 and for FIG. 8B for P4 where n=2).

FIGS. 9A-9D provide dynamic light scattering plots of drug loaded micelles of P1 (FIG. 9A) and P2 (FIG. 9B)(10 mg/mL) with 4 mg/mL paclitaxel and unloaded micelles of P3 (5 mg/mL) in the presence (FIG. 9D) and absence (FIG. 9C) of 5 mg/mL BSA.

FIG. 10A is a graph of MCF7/ADR cell viability after 24 hour incubation with P1-P4 at concentrations of up to 20 mg/mL. FIGS. 10B and 10C are graphs of MCF7 and MDCK cell viability, respectively, after 2 hour incubation with P1-P4 at concentrations of up to 20 mg/mL.

FIGS. 11A and 11B are graphs of flow cytometric analyses of MCF7/ADR cells after 60 minute incubation with Atto425-labeled P4 and P5, respectively, at 37° C. and various concentrations. FIG. 11C is a graph of a flow cytometric analysis of MCF7 cells after a 60 minute incubation with Atto425-labeled P5 at 37° C. and various concentration. FIGS. 11D and 11E are graphs of flow cytometric analyses of MCF7/ADR cells after incubation for different time intervals with Atto425-labeled P4 and P5, respectively, at 37° C. FIG. 11F is a graph of a flow cytometric analysis of MCF7/ADR cells after incubation for 60 minutes with Atto425-labeled P4 at 37° C. and 4° C. at a concentration of 0.1 mg/mL.

FIGS. 12A-12C are confocal micrographs of MCF7/ADR cells after a 5 minute (FIG. 12B) or 60 minute (FIGS. 12A and 12C) incubation with Atto425-labeled P4 (FIGS. 12B and 12C) or P5 (FIG. 12A) at 37° C. at a concentration of 0.2 mg/mL, λₑₓ=405 nm, band pass filter 420/60 nm, magnification 63×. FIGS. 12D-12F provide a Z-stack obtained from confocal microscopy of MCF7/ADR cells after 5 minute incubation with Atto425-labeled P4 at 37° C. at a concentration of 0.2 mg/mL.

FIG. 12D represents blue fluorescence picture (λₑₓ=405 nm, band pass filter 420/60 nm), FIG. 12E represents differential interference contrast (DIC), and FIG. 12F gives the orthogonal view of the same z-stack. Slices are separated by 1 μm, bars represent 20 μm, magnification 63×.

FIGS. 13A-13C demonstrate paclitaxel dose dependent viability of multi-drug resistant MCF7/ADR cells. FIG. 13A provides a comparison of P2 and P3 formulated paclitaxel. FIG. 13B demonstrates no change in paclitaxel activity is observed after freeze-drying and reconstitution in deionized water (shown here with P4).

FIG. 14 shows relative tumor weights (FIG. 14A) and tumor inhibition (FIG. 14B) in mice comparing negative controls, treatment with compositions according to the invention, and a commercial product.

FIG. 15A provides a reaction scheme for a preparation of star-block copolymers. FIG. 15B provides a schematic of a preparation of a bi-functional initiator for the two step preparation of triblock copolymers (Witte et al. (1974) Liebigs Ann. Chem., 6:996; Kobayashi et al. (1987) Macromol., 20:1729).

### DETAILED DESCRIPTION OF THE INVENTION

The instant invention allows for the solubilization of compounds (e.g., hydrophobic drugs) in aqueous solutions (e.g., water, blood). A number of highly potent drugs are not soluble in water and are, therefore, difficult to deliver to the human body. The instant invention utilizes highly water soluble and nontoxic polymers to incorporate these kinds of drugs (e.g., paclitaxel) into micelles formed by the polymer. The presence of the polymers increases the solubility in water and aqueous solutions by orders of magnitude. This allows for largely increased dose administration to patients and would be particularly beneficial in the treatment of various diseases such as cancer.

As stated above, a wide variety of highly active drugs suffer from very low solubility in aqueous media. This is a major limitation in their use as orally or intravenously administered drugs. Numerous polymers, in particular amphiphilic block copolymers have been studied in order to find a suitable polymer carrier system for hydrophobic drugs. In particular, solubilization of the hydrophobic macrocycle paclitaxel (solubility in water approx. 0.3 μg/ml), widely used in cancer chemotherapy has been investigated herein. ABA-type block copoly(2-oxazoline)s (also termed poly(N-acetylethylenimine)s) of the instant invention consisting of hydrophilic A blocks (e.g., 2-methyl-2-oxazoline) and hydrophobic B blocks (e.g., consisting of 2-butyl-2-oxazoline or 2-nonyl-2-oxazoline) are extraordinarily well suited to solubilize high amounts of paclitaxel in aqueous media at physiologically relevant pH.

Only a quite limited number of types of polymers are widely recognized as suitable for a wide range of biomedical materials. Problems with these polymers include a lack of chemical and structural versatility and definition. Poly(2-oxazoline)s are a very valuable novel alternative for biomedical materials in general and as drug carriers in particular. The defined cationic ring opening polymerization reaction and chemical versatility of poly(2-oxazoline)s allows for very exact tuning of their solubility, their thermal responsiveness (LCST), and their aggregation behavior in aqueous solutions. Depending on the side chain, poly(2-oxazoline)s or poly(2-oxazoline) blocks can be extremely hydrophilic, amphiphilic, hydrophobic, or fluorophilic. Additionally, a wide range of side chain moieties have been introduced, including carboxylic acids, amines, aldehydes, alkynes and thiols. These allow a wide range of specific coupling reactions (chemoselective ligations) with bioactive compounds, e.g. peptides or drugs. In addition, multi-block, star-like, and star-like block copolymers may be synthesized.

The preparation of compound (e.g., paclitaxel) loaded poly(2-oxazoline) loaded micelles is facile via a thin film method. Briefly, both polymer and the drug (e.g., paclitaxel) are dissolved in acetonitrile, a common solvent for both compounds. The solvent is removed in a stream of gas (nitrogen or air). In order to remove possible residual solvent, the films are objected to vacuum (approx. 0.2 mbar) overnight or at least three hours. Subsequently, the desired aqueous media is added (e.g., water or pH 7.4 buffer solution such as phosphate buffered saline) and the polymer drug film is solubilized by vortexing or gentle shaking. At certain drug-polymer ratios, solubilization is facilitated at 37° C. After filtration (pore size 0.22-0.45 μm) to remove eventually non-dissolved paclitaxel particles or precipitated drug-polymer aggregates, the aqueous micellar drug formulation can be analyzed to determine the final drug concentration by high performance liquid chromatography (HPLC). The HPLC analysis was performed under isocratic conditions with a solvent mixture of 45% water and 55% acetonitrile and the amount of paclitaxel was determined using a calibration curve.

It is shown herein that various poly(2-oxazoline)s, differing in molecular weight, polymer architecture, and block lengths, are excellent solubilizers for drugs such as paclitaxel at polymer concentrations ranging from 0.2 wt % to 1% wt. and paclitaxel concentrations up to 8.3 mg/ml in 1 wt. % polymer solutions (10 mg/ml) can be obtained. This value is about 28,000 times the normal solubility of paclitaxel in water and greatly exceeds any solubilization potential in comparable polymer concentrations in aqueous solutions of any compound. The final loading capacity of the micelles was thus as high as 45% (w/w). Sizes of the drug-polymer micelles vary depending on the drug loading and the polymer used, but are typically found around 20-23 nm with very narrow size distribution (PDI ≦0.1). This size range is well suited for intravenous administration. The size of the formed particles was also confirmed by atomic force microscopy.

Furthermore, these formulations were investigated towards their behavior after freeze drying and reconstitution in water. It was found that this process did not alter the amount of paclitaxel found and also the size of the aggregates did not change significantly. Such characteristics are preferable for commercialization since it is desirable to supply dry powders as opposed to micellar solutions, which are much more likely to undergo aging processes. Importantly, the incorporated drug retains its toxicity towards cancer cells. This is in stark contrast to other polymers which have failed to properly release the incorporated drug and renders the incorporated drug inactive.

These results are unexpected as 2-oxazoline polymers were not designed for drug formulations and most 2-oxazoline polymers have a relatively high overall hydrophilicity. Moreover, during measurements for the critical micellar concentration (CMC) by pyrene probe assay, it was determined that the micellar core forms a relatively polar environment. It was not expected that a polar and well hydrated micellar core would incorporate significant amounts of highly hydrophobic drug.

In addition to paclitaxel, other relevant hydrophobic drugs which significantly vary in their chemical nature have been successfully incorporated in these micelles. For example, cyclosporine A (a cyclic peptide and powerful immunosuppressant) and amphotercin B (a polyene polyole macrolactone (an antifungal agent which can be used against systemic fungal infections in immunocompromised patients)) have been incorporated into the polymers of the instant invention.

The described invention utilizes less material to solubilize the same amount of bioactive substance, e.g., paclitaxel. While a 10% solution (v/v) of Cremophor EL®/EtOH is needed to solubilize 600 μg/mL paclitaxel in aqueous solution, this is possible to achieve with only a 0.2% solution (w/w) of the described polymers. This significantly reduces the additional load of substances given to patients and is expected to minimize eventual side effects. Additionally, reduced side effects will occur because the polymers described in this invention are not known to be toxic or hazardous in any way in a relevant concentration range. Furthermore, the described paclitaxel-poly(2-oxazoline) formulations are easy to prepare and can be freeze-dried and easily reconstituted by addition of the desired parenteral administration solution (e.g., saline for i.v. injection). Storage as a solid also typically enhances shelf-life of bioactive components.

Highly water soluble, well-defined poly(2-methyl-2-oxazoline) and poly(2-ethyl-2-oxazoline) polymers have been shown to not undergo unspecific accumulation in a host and the polymers are very rapidly excreted via the kidneys in the mouse. Furthermore, no cytotoxicity in various cell types of human, canine, and murine origin has been generally observed, even at very high concentrations of up to 20 mg/mL. Concentration, time and temperature dependent studies of cellular uptake reveal that, depending on then polymer structure, the cellular uptake can occur extremely fast and very efficiently, even at very low concentrations. Furthermore, the cellular uptake of poly(2-oxazoline)s is typically energy dependent, as at 4° C. no cellular uptake was observed for most polymer structures. In conclusion, the structural and chemical versatility of poly(2-oxazoline)s, together with their excellent biocompatibility, make this class of polymer ideal for delivering drugs and biomaterials.

Surprisingly, it has been demonstrated herein that biocompatible, water soluble polymers comprising at least one hydrophobic block of poly(2-oxazoline)s with hydrophobic side chains form compositions with large amounts of highly hydrophobic drugs (40% w/w), even at polymer concentrations as low as 0.2% (w/v).

### I. Definitions

The following definitions are provided to facilitate an understanding of the present invention:

As used herein, the term “lipophilic” refers to the ability to dissolve in lipids. “Hydrophobic” designates a preference for apolar environments (e.g., a hydrophobic substance or moiety is more readily dissolved in or wetted by non-polar solvents, such as hydrocarbons, than by water).

As used herein, the term “hydrophilic” means the ability to dissolve in water.

As used herein, the term “amphiphilic” means the ability to dissolve in both water and lipids/apolr environments. Typically, an amphiphilic compound comprises a hydrophilic portion and a lipophilic (hydrophobic) portion.

As used herein, the term “biocompatible” refers to a substance which produces no significant untoward effects when applied to, or administered to, a given organism.

As used herein, aqueous environments, aqueous media, aqueous solutions or the like refer to solvent systems wherein 50% (v/v) or more, preferably 70% or more, more preferably 90% or more and in particular substantially 100% of the total volume of solvent(s) is water.

As used herein, the term “polymer” denotes molecules formed from the chemical union of two or more repeating units or monomers. The term “block copolymer” most simply refers to conjugates of at least two different polymer segments, wherein each polymer segment comprises two or more adjacent units of the same kind.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, or the addition of stabilizers.

“Polypeptide” and “protein” are sometimes used interchangeably herein and indicate a molecular chain of amino acids. The term polypeptide encompasses peptides, oligopeptides, and proteins. The terms also include post-expression modifications of the polypeptide, for example, glycosylations, acetylations, phosphorylations and the like. In addition, protein fragments, analogs, mutated or variant proteins, fusion proteins and the like are included within the meaning of polypeptide.

The term “isolated” may refer to protein, nucleic acid, compound, or cell that has been sufficiently separated from the environment with which it would naturally be associated, so as to exist in “substantially pure” form. “Isolated” does not necessarily mean the exclusion of artificial or synthetic mixtures with other compounds or materials, or the presence of impurities that do not interfere with the fundamental activity, and that may be present, for example, due to incomplete purification.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), bulking substance (e.g., lactose, mannitol), excipient, auxiliary agent, filler, disintegrant, lubricating agent, binder, stabilizer, preservative or vehicle with which an active agent of the present invention is administered. Pharmaceutically acceptable carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. The compositions can be incorporated into particulate preparations of polymeric compounds such as polylactic acid, polyglycolic acid, etc., or into liposomes or micelles. Such compositions may influence the physical state, stability, rate of in vivo release, and rate of in vivo clearance of components of a pharmaceutical composition of the present invention. The pharmaceutical composition of the present invention can be prepared, for example, in liquid form, or can be in dried powder form (e.g., lyophilized). Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, 20th Edition, (Lippincott, Williams and Wilkins), 2000; Liberman, et al., Eds., Pharmaceutical Dosage Forms, Marcel Decker, New York, N.Y., 1980; and Kibbe, et al., Eds., Handbook of Pharmaceutical Excipients (3rd Ed.), American Pharmaceutical Association, Washington, 1999.

The term “alkyl,” as employed herein, includes both straight and branched chain hydrocarbons containing about 1 to about 50 carbons, about 1 to about 20, about 1 to about 15, or about 1 to about 10 carbons in the main chain. The hydrocarbon chain may be saturated or unsaturated (i.e., comprise double and/or triple bonds). The hydrocarbon chain may also be cyclic or comprise a portion which is cyclic. The hydrocarbon chain of the alkyl groups may be interrupted with heteroatoms such as oxygen, nitrogen, or sulfur atoms. Each alkyl group may optionally be substituted with substituents which include, for example, alkyl, halo (such as F, Cl, Br, I), haloalkyl (e.g., CCl₃ or CF₃), alkoxyl, alkylthio, hydroxy, methoxy, carboxyl, oxo, epoxy, alkyloxycarbonyl, alkylcarbonyloxy, amino, carbamoyl (e.g., NH₂C(═O)— or NHRC(═O)—, wherein R is an alkyl), urea (—NHCONH₂), alkylurea, aryl, ether, ester, thioester, nitrile, nitro, amide, carbonyl, carboxylate and thiol. Examples of simple alkyls include, without limitation, propyl, butyl, pentyl, hexyl, heptyl, octyl and nonyl.

The term “aryl,” as employed herein, refers to monocyclic and bicyclic aromatic groups containing 6 to 10 carbons in the ring portion. Aryl groups may be optionally substituted through available carbon atoms. The aromatic ring system may include heteroatoms such as sulfur, oxygen, or nitrogen.

### II. Polymer

In a preferred embodiment of the instant invention, the synthetic polymers of the complexes are block copolymers. More specifically, the synthetic polymers are block copolymers which comprise at least one hydrophilic polymer segment and at least one hydrophobic (lipophilic) polymer segment. Block copolymers are most simply defined as conjugates of at least two different polymer segments (Tirrel, M. In: Interactions of Surfactants with Polymers and Proteins. Goddard E. D. and Ananthapadmanabhan, K. P. (eds.), CRC Press, Boca Raton, Ann Arbor, London, Tokyo, pp. 59-122, 1992). The simplest block copolymer architecture contains two segments joined at their termini to give an A-B type diblock. Consequent conjugation of more than two segments by their termini yields A-B-A type triblock, A-B-A-B-type multiblock, or even multisegment A-B-C-architectures. If a main chain in the block copolymer can be defined in which one or several repeating units are linked to different polymer segments, then the copolymer has a graft architecture of, e.g., an A(B)ₙ type. More complex architectures include for example (AB)ₙ (wherein m is about 1 to about 100) or AₙBₘ starblocks which have more than two polymer segments linked to a single center. An exemplary block copolymer of the instant invention has the formula A-B or B-A, wherein A is a hydrophilic polymer segment and B is a hydrophobic polymer segment. Another exemplary block copolymer has the formula A-B-A. Block copolymers structures include, without limitation, linear copolymers, star-like block copolymers, graft block copolymers, dendrimer based copolymers, and hyperbranched (e.g., at least two points of branching) block copolymers. The segments of the block copolymer may have from about 2 to about 1000, about 2 to about 300, or about 2 to about 100 repeating units or monomers.

Well-defined poly(2-oxazoline) block copolymers of the instant invention may be synthesized by the living cationic ring-opening polymerization of 2-oxazolines. The synthetic versatility of poly(2-oxazoline)s allows for a precise control over polymer termini and hydrophilic-lipophilic balance (HLB). Block length, structure, charge, and charge distribution of poly(2-oxazoline)s may be varied. For example, the size of the hydrophilic and/hydrophobic blocks may be alteres, triblock polymers may be synthesized, star-like block copolymers may be used, polymer termini may be altered, and ionic side chains and/or ionic termini may also be incorporated. Ionic side chains (e.g., comprising —R—NH₂ or R—COOH, wherein R is an alkyl) may be incorporated into the hydrophilic (preferably) or hydrophobic block.

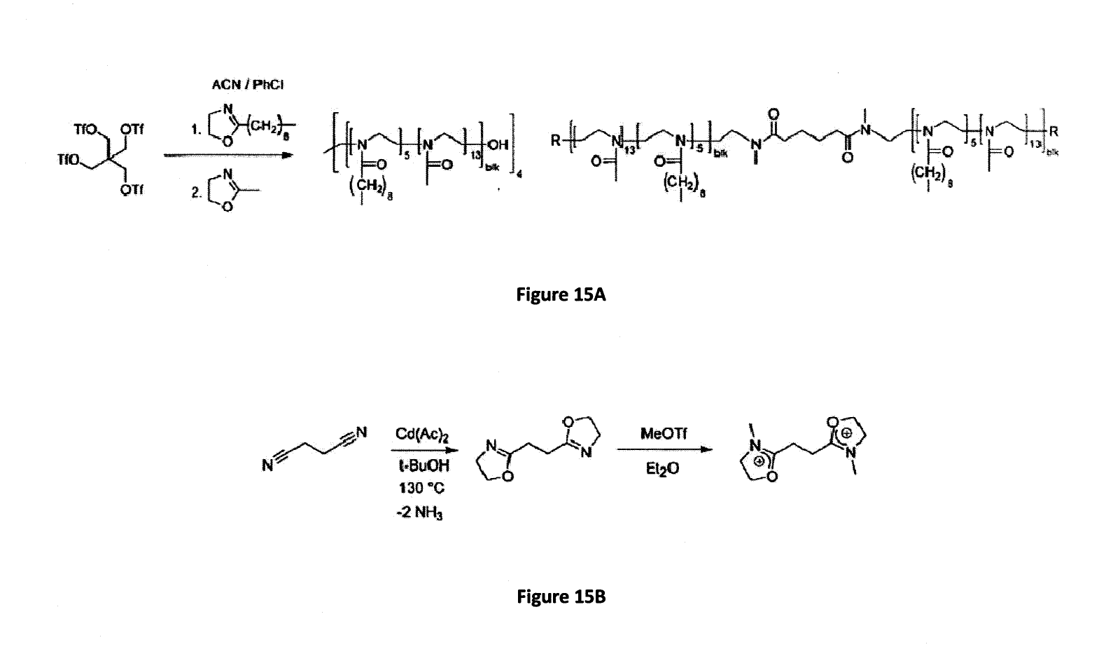

Poly(2-oxazoline)s (also known as 2-substituted 4,5-dihydro oxazoles) are polysoaps and depending on the residue at the 2-position of the monomer can be hydrophilic (e.g., methyl, ethyl) or hydrophobic (e.g. propyl, pentyl, nonyl, phenyl, and the like) polymers. Moreover, numerous monomers introducing pending functional groups are available (Taubmann et al. (2005) Macromol. Biosci., 5:603; Cesana et al. (2006) Macromol. Chem. Phys., 207:183; Luxenhofer et al. (2006) Macromol., 39:3509; Cesana et al. (2007) Macromol. Rapid Comm., 28:608). Poly(2-oxazoline)s can be obtained by living cationic ring-opening polymerization (CROP), resulting in well-defined block copolymers and telechelic polymers of narrow polydispersities (Nuyken, et al. (1996) Macromol. Chem. Phys., 197:83; Persigehl et al. (2000) Macromol., 33:6977; Kotre et al. (2002) Macromol. Rapid Comm., 23:871; Fustin et al. (2007) Soft Matter, 3:79; Hoogenboom et al. (2007) Macromol., 40:2837). Several reports suggest that hydrophilic poly(2-oxazoline)s are essentially non-toxic and biocompatible (Goddard et al. (1989) J. Control. Release, 10:5; Woodle et al. (1994) Bioconjugate Chem., 5:493; Zalipsky et al. (1996) J. Pharm. Sci., 85:133; Lee et al. (2003) J. Control. Release, 89:437; Gaertner et al. (2007) J. Control. Release, 119:291). Using lipid triflates or pluritriflates, lipopolymers (Nuyken, et al. (1996) Macromol. Chem. Phys., 197:83; Persigehl et al. (2000) Macromol., 33:6977; Kotre et al. (2002) Macromol. Rapid Comm., 23:871; Fustin et al. (2007) Soft Matter, 3:79; Hoogenboom et al. (2007) Macromol., 40:2837; Punucker et al. (2007) Soft Matter, 3:333; Garg et al. (2007) Biophys. J., 92:1263; Punucker et al. (2007) Phys. Rev. Lett., 98:078102/1; Luedtke et al. (2005) Macromol. Biosci., 5:384; Purmcker et al. (2005) J. Am. Chem. Soc., 127:1258) or star-like poly(2-oxazoline)s (FIG. 15A) are readily accessible. Additionally, various poly(2-oxazoline)s with terminal quaternary amine groups have been reported, which interact strongly with bacterial cell membranes (Waschinski et al. (2005) Macromol. Biosci., 5:149; Waschinski et al. (2005) Biomacromol., 6:235).

In a particular embodiment, the biocompatible, water soluble copolymer of the instant invention comprises at least one hydrophilic block A and at least one hydrophobic block B. The at least one hydrophilic block A and at least one hydrophobic block B are attached through linkages which are stable or labile (e.g., biodegeradable under physiological conditions (e.g., by the action of biologically formed entities which can be enzymes or other products of the organism)). Although the hydrophilic block of the polymer preferably comprises at least one poly(2-oxazoline), the hydrophilic block may also comprise at least one polyethyleneoxide, polyester, or polyamino acid (e.g. poly(glutamic acid) or poly(aspartic acid)) or block thereof. The hydrophobic block may comprise a hydrophobic poly(2-oxazoline). Examples of hydrophilic poly(2-oxazoline)s include, without limitation, 2-methyl-2-oxazoline, 2-ethyl-2-oxazoline, and mixtures thereof. The degree of polymerization may vary between 5 and 500. Examples of the hydrophobic polymer block include poly(2-oxazoline)s with hydrophobic substituents at the 2-position of the oxazoline ring. In a particular embodiment, the hydrophobic substituent is an alkyl or an aryl. In another embodiment, the hydrophobic substituent comprises 3 to about 50 carbon atoms, 3 to about 20 carbon atoms, 3 to about 12 carbon atoms, particularly 3 to about 6 carbon atoms, or 4 to about 6 carbons. In a particular embodiment, the hydrophobic block copolymer is 2-butyl-2-oxazoline, 2-propyl-2-oxazoline, or mixtures thereof. The hydrophobic block may consist of 1-300 monomer units. In a particular embodiment, the ratio of repeating hydrophilic units to repeating hydrophobic units (in terms of the numbers of repeating units) typically ranges from about 20:1 to 1:2, preferably from about 10:1 to 1:1, and more preferably from about 7:1 to 3:1.

In a particular embodiment of the instant invention, the copolymer of the instant invention is represented by the formula:

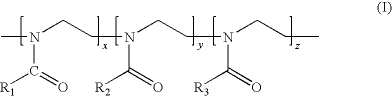

wherein x and y are independently selected between 1 and about 300, particularly about 5 to about 150, and more particularly about 10 to about 100; z is either 0 or from between 1 and about 300, particularly about 5 to about 150, and more particularly about 10 to about 100; R₁ and R₃ are independently selected from the group consisting of —H, —OH, —NH₂, —SH, —CH₃, —CH₂CH₃, and an alkyl comprising 1 or 2 carbon atoms; and R₂ is selected from the group consisting of an alkyl or an aryl. In a particular embodiment, x, y, and z are independently 5 or more, 10 or more, or 20 or more, and preferably less than 300, less than 200, less than 100, or less than 50. In a particular embodiment, R₁ and R₃ are independently selected from the group consisting of —CH₃ and —CH₂CH₃. In a particular embodiment, R₂ is the formula (CH₂)ₙ—R₄, wherein R₄ is —OH, —COOH, —CHCH₂, —SH, —NH₂, —CCH, —CH₃, or —CHO and wherein n is about 2 to about 50, about 2 to about 20, about 2 to about 12, or about 3 to 6. In a particular embodiment, R₂ comprises 3 to about 50 carbon atoms, 3 to about 20 carbon atoms, 3 to about 12 carbon atoms, or 3 to about 6 carbon atoms. In yet another embodiment, R₂ is butyl (including isopropyl, sec-butyl, or tert-butyl) or propyl (including isopropyl). In yet another embodiment, R₂ is —CH₂—CH₂—CH₂—CH₃ or —CH₂—CH₂—CH₃.

The polymers of the instant invention increase the solubility of hydrophobic drugs by a number of orders of magnitude using as little as 1% (w/w, i.e. 10 mg/mL) of amphiphilic block copolymers in water or aqueous solutions. Extremely high loading capacities (loading capacity=(mass of hydrophobic compound)/(mass of polymer compound plus hydrophobic compound)*100%)) such as >40% (w/w), can be achieved. The high loading capacities at relatively low polymer concentration allow, in contrast to other commercialized systems, the preparation of formulations of low viscosity but high drug content. At the same time, there is a significant reduction in the amount of solubilizer subjects receive upon parenteral administration, thereby reducing the risk of adverse health effects.

Furthermore, the instant polymers exhibit a loading efficiency (i.e. (amount of solubilized hydrophobic compound/amount of initially charged hydrophobic compound)*100%) that can reach 100% and are generally found to be very high (>80%). This is a significant finding as high loading efficiencies are of importance for commercial applications for the reduction of production costs.

The polymers of the instant invention may be utilized to solubilize highly hydrophobic bioactive substances of a solubility of <1 mg/mL, preferably <0.1 mg/mL or <0.01 mg/mL in water or aqueous media in a pH range of 0-14, preferably between pH 4 and 10. The preparation of the solutions of polymer and hydrophobic drug may be performed as follows: The amphiphilic block copolymer may be dissolved together with the hydrophobic compound in a common solvent, e.g. acetonitrile or dimethylsulfoxide. After removal of the solvent (e.g. by a stream of inert gas, gentle heating and/or application of reduced pressure) the films formed by the polymer and the hydrophobic compound can be easily dissolved in water or the desired aqueous solution and are tempered at elevated temperatures. The formed compositions form aggregates of sizes between 5 and 200 nm, preferably between 5 and 100 nm. The compositions can be freeze-dried from water or aqueous solutions and reconstituted in water or aqueous solutions without compromising loading capacities or particle sizes.

Amphiphilic block copolymers can be obtained from hydrophilic 2-methyl-2-oxazoline (MeOx) and hydrophobic 2-nonyl-2-oxazoline (NonOx) (Bonne et al. (2004) Colloid Polym. Sci., 282:833; Bonne et al. (2007) Coll. Polym. Sci., 285:491). Various amphiphilic block copolymers (also additionally bearing carboxylic acid side chains for micellar catalysis (Zarka et al. (2003) Chem-Eur. J., 9:3228; Bortenschlager et al. (2005) J. Organomet. Chem., 690:6233; Rossbach et al. (2006) Angew. Chem. Int. Ed., 45:1309)) and lipopolymers have been reported and their aggregation behavior in aqueous solution was studied (Bonne et al. (2004) Colloid Polym. Sci., 282:833; Bonne et al. (2007) Coll. Polym. Sci., 285:491). CROP allows for an exact tuning of the hydrophilic-lipophilic balance (HLB) and initiation with a bi-functional initiator allows two step synthesis of triblock copolymers (FIG. 15B) in contrast to the three step synthesis necessary when, e.g., methyltriflate is used as an initiator. This approach has the additional benefit that both polymer termini can be easily functionalized with the same moiety.

The initiators used to generate the copolymers of the instant invention can be any initiator used in the art. Additionally, the termini of the copolymers of the instant invention can be any terminus known in the art. The polymers can be prepared from mono-, bi- or multifunctional initiators (such as multifunctional triflates or multifunctional oxazolines) such as, but not restricted to, methyltriflate, 1,2-bis(N-methlyoxazolinium triflate) ethane or pentaerithritol tetrakistriflate. Examples of polymer termini include, for example, —OH, —OCH₃,

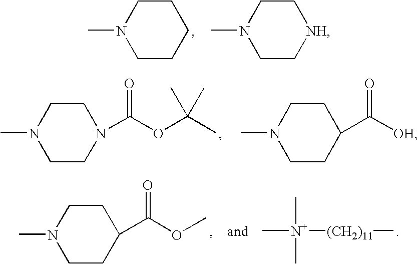

Amphiphilic copolymers of the instant invention (e.g., piperazine terminated copolymers) may be additionally labeled with a fluorescent dye (e.g., fluorescein isothiocyanate, FITC) to allow evaluation of the localization (e.g. in plasma membrane compartments such as lipid rafts, caveolae, clathrin coated pits) of these polymers by confocal microscopy (Batrakova et al. (2001) J. Pharmacol. Exp. Ther., 299:483; Bonne et al. (2004) Colloid Polym. Sci., 282:833; Bonne et al. (2007) Coll. Polym. Sci., 285:491).

The preferred size of the complexes is between about 5 nm and about 500 nm, between about 5 and about 200 nm, between about 10 and about 150 nm, between about 10 nm and about 100 nm, or about 10 nm and about 50 nm. The complexes do not aggregate and remain within the preferred size range for at least 1 hour after dispersion in the aqueous solution at the physiological pH and ionic strength, for example in phosphate buffered saline, pH 7.4. The sizes may be measured as effective diameters by dynamic light scattering (see, e.g., Batrakova et al. (2007) Bioconjugate Chem., 18:1498-1506). It is preferred that, after dispersion in aqueous solution, the complexes remain stable, i.e., do not aggregate and/or precipitate for at least 2 hours, preferably for 12 hours, still more preferably for 24 hours (e.g., at room temperature, preferably at elevated temperatures (e.g., 37° C. or 40° C.). In a aparticualr embodiment, the copolymers may have a number average molecular weight (Mn) (e.g., as determined by gel permeation chromatography) ranging from about 3 to about 30, from about 4 to about 25, or from about 6 to about 20 kg/mol. In yet another embodiment, the polydispersities (PDI) is below 1.3, below 1.25, below 1.1, or can be as low as 1.001. In still another embodiment of the instant invention, the aggregates (micelles) formed by the polymers of the instant invention have a critical micelle concentration (cmc) which are less than 250 mg/L, particularly from about 5 mg/L to about 150 mg/mL or from about 5 to about 100 mg/L.

The instant invention also encompasses compositions comprising the polymer of the instant invention and at least one pharmaceutically acceptable carrier. The composition may further comprise at least one bioactive agent (e.g. therapeutic agent and/or diagnostic agent) as set forth below.

### III. Bioactive and Therapeutic Agents

The polymers of the instant invention may be used to deliver any agent(s) or compound(s), particularly bioactive agents (e.g., therapeutic agent or diagnostic agent) to a subject (including non-human animals). As used herein, the term “bioactive agent” also includes compounds to be screened as potential leads in the development of drugs or plant protecting agents. Indeed, the instant invention encompasses methods for the detection of active compounds which interact with a target of interest in a screening test comprising incorporating an active compound into a composition of the instant invention and subjecting the composition to the screening test. In one embodiment, fungicides, pesticides, insecticides, herbicides, any further compounds suitable in the field of plant or crop protection such as phytohormones, may be delivered with the polymers of the instant invention.

The bioactive agent, particularly therapeutic agents, of the instant invention include, without limitation, polypeptides, peptides, glycoproteins, nucleic acids, synthetic and natural drugs, peptoides, polyenes, macrocyles, glycosides, terpenes, terpenoids, aliphatic and aromatic compounds, and their derivatives. In a preferred embodiment, the therapeutic agent is a chemical compound such as a synthetic and natural drug. In another preferred embodiment, the therapeutic agent effects amelioration and/or cure of a disease, disorder, pathology, and/or the symptoms associated therewith. The polymers of the instant invention may encapsulate one or more therapeutic agents.

Preferably, the therapeutic agent is hydrophobic. Therapeutic agents that may be solubilized or dispersed by the polymers of the present invention can be any bioactive agent and particularly those having limited solubility or dispersibility in an aqueous or hydrophilic environment, or any bioactive agent that requires enhanced solubility or dispersibility. In a particular embodiment, the polymers of the instant invention may be utilized to solubilize highly hydrophobic bioactive substances having a solubility of <1 mg/mL, <0.1 mg/mL, <50 μg/ml, or <10 μg/mL in water or aqueous media in a pH range of 0-14, preferably between pH 4 and 10, particularly at 20° C. Suitable drugs include, without limitation, those presented in Goodman and Gilman's The Pharmacological Basis of Therapeutics (9th Ed.) or The Merck Index (12th Ed.). Genera of drugs include, without limitation, drugs acting at synaptic and neuroeffector junctional sites, drugs acting on the central nervous system, drugs that influence inflammatory responses, drugs that affect the composition of body fluids, drugs affecting renal function and electrolyte metabolism, cardiovascular drugs, drugs affecting gastrointestinal function, drugs affecting uterine motility, chemotherapeutic agents e.g., for cancer, for parasitic infections, and for microbial diseases), antineoplastic agents, immunosuppressive agents, drugs affecting the blood and blood-forming organs, hormones and hormone antagonists, dermatological agents, heavy metal antagonists, vitamins and nutrients, vaccines, oligonucleotides and gene therapies. Examples of drugs suitable for use in the present invention include, without limitation, testosterone, testosterone enanthate, testosterone cypionate, methyltestosterone, amphotericin B, nifedipine, griseofulvin, taxanes (including, without limitation, paclitaxel, docetaxel, larotaxel, ortataxel, tesetaxel and the like), doxorubicin, daunomycin, indomethacin, ibuprofen, etoposide, cyclosporin A, vitamin E, and testosterone. In a particular embodiment, the drug is nifedipine, griseofulvin, a taxane, amphotericin B, etoposide or cyclosporin A.

In a particular embodiment, the hydrophobic therapeutic agent and amphiphilic block copolymer of the instant invention are in a weight ratio may be 1:20 or higher (e.g., 1:10). The weight ration may be at least 1:9, at least 2:8, at least 3:7, or at least 4:6. Typically the weight ratio is less than 4:5 or 1:1. In another embodiment, the polymer has a drug load (i.e. a ratio of the weight of the bioactive agent to the sum of the weights of the active agent and the block copolymer) of 25% or more, 30% or more, 35% or more, or 40% or more.

### IV. Administration

The polymer-therapeutic agent complexes described herein will generally be administered to a patient as a pharmaceutical preparation. The term “patient” as used herein refers to human or animal subjects. These polymer-therapeutic agent complexes may be employed therapeutically, under the guidance of a physician. While the therapeutic agents are exemplified herein, any bioactive agent may be administered to a patient, e.g., a diagnostic agent.

The compositions comprising the polymer-therapeutic agent complex of the instant invention may be conveniently formulated for administration with any pharmaceutically acceptable carrier(s). For example, the complexes may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the polymer-therapeutic agent complexes in the chosen medium may be varied and the medium may be chosen based on the desired route of administration of the pharmaceutical preparation. Except insofar as any conventional media or agent is incompatible with the polymer-therapeutic agent complexes to be administered, its use in the pharmaceutical preparation is contemplated.

The dose and dosage regimen of polymer-therapeutic agent complexes according to the invention that are suitable for administration to a particular patient may be determined by a physician considering the patient's age, sex, weight, general medical condition, and the specific condition for which the polymer-therapeutic agent complex is being administered and the severity thereof. The physician may also take into account the route of administration, the pharmaceutical carrier, and the polymer-therapeutic agent complex's biological activity.

Selection of a suitable pharmaceutical preparation will also depend upon the mode of administration chosen. For example, the polymer-therapeutic agent complex of the invention may be administered by direct injection to a desired site. In this instance, a pharmaceutical preparation comprises the polymer-therapeutic agent complex dispersed in a medium that is compatible with the site of injection.

Polymer-therapeutic agent complexes of the instant invention may be administered by any method. For example, the polymer-therapeutic agent complex of the instant invention can be administered, without limitation parenterally, subcutaneously, orally, topically, pulmonarily, rectally, vaginally, intravenously, intraperitoneally, intrathecally, intracerbrally, epidurally, intramuscularly, intradermally, or intracarotidly. In a particular embodiment, the complexes are administered intravenously or intraperitoneally. Pharmaceutical preparations for injection are known in the art. If injection is selected as a method for administering the polymer-therapeutic agent complex, steps must be taken to ensure that sufficient amounts of the molecules or cells reach their target cells to exert a biological effect. Dosage forms for oral administration include, without limitation, tablets (e.g., coated and uncoated, chewable), gelatin capsules (e.g., soft or hard), lozenges, troches, solutions, emulsions, suspensions, syrups, elixirs, powders/granules (e.g., reconstitutable or dispersible) gums, and effervescent tablets. Dosage forms for parenteral administration include, without limitation, solutions, emulsions, suspensions, dispersions and powders/granules for reconstitution. Dosage forms for topical administration include, without limitation, creams, gels, ointments, salves, patches and transdermal delivery systems.

Pharmaceutical compositions containing a polymer-therapeutic agent complex of the present invention as the active ingredient in intimate admixture with a pharmaceutically acceptable carrier can be prepared according to conventional pharmaceutical compounding techniques. The carrier may take a wide variety of forms depending on the form of preparation desired for administration, e.g., intravenous, oral, direct injection, intracranial, and intravitreal.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of polymer-therapeutic agent complexes may be determined by evaluating the toxicity of the molecules or cells in animal models. Various concentrations of polymer-therapeutic agent complexes in pharmaceutical preparations may be administered to mice, and the minimal and maximal dosages may be determined based on the beneficial results and side effects observed as a result of the treatment. Appropriate dosage unit may also be determined by assessing the efficacy of the polymer-therapeutic agent complex treatment in combination with other standard drugs. The dosage units of polymer-therapeutic agent complex may be determined individually or in combination with each treatment according to the effect detected.

The pharmaceutical preparation comprising the polymer-therapeutic agent complexes may be administered at appropriate intervals, for example, at least twice a day or more until the pathological symptoms are reduced or alleviated, after which the dosage may be reduced to a maintenance level. The appropriate interval in a particular case would normally depend on the condition of the patient.

In a particular embodiment, the polymer-therapeutic agent is administered to a cell of the body in an isotonic solution at physiological pH 7.4. However, the complexes can be prepared before administration at a pH below or above pH 7.4.

The instant invention encompasses methods of treating or diagnosing a disease/disorder comprising administering to a subject in need thereof a composition comprising a polymer-bioactive agent complex of the instant invention and, preferably, at least one pharmaceutically acceptable carrier. In a particular embodiment, the disease is cancer and the polymer comprises at least one chemotherapeutic agent (particularly a taxane (e.g., paclitaxel). Other methods of treating the disease or disorder may be combined with the methods of the instant invention (e.g., other chemotherapeutic agents or therapy (e.g., radiation) may be co-administered with the compositions of the instant invention.

The following examples provide illustrative methods of practicing the instant invention, and are not intended to limit the scope of the invention in any way.

### Example 1

### Preparation of Methyl-P [MeOx₂₆-b-BUOx₂₀-b-MeOx₂₈]-piperidine (LXRB20)

Methyltriflate (24.7 mg, 0.150 mmol, 1 eq) and 334 mg 2-methyl-2-oxazoline (3.9 mmol, 26 eq) were dissolved in 3.14 mL (2.45 g) acetonitrile. The mixture was heated to 130° C. for 20 minutes using a microwave. After cooling to room temperature, 136 mg (5% w/w) of the reaction mixture was removed for analysis of the first block with nuclear magnetic resonance (NMR) and gel permeation chromatography (GPC). After addition of 364.4 mg 2-butyl-2-oxazoline (2.87 mmol, 20 eq), the mixture was again heated to 130° C. for 20 minutes. Once more, after removal of an aliquot (306.9 mg, 10% w/w) was removed, 306.9 mg MeOx (3.6 mmol, 28 eq) was added and the mixture was heated to 130° C. for 20 minutes. After cooling to room temperature (RT), 80 μL of piperidine was added and the mixture was stirred overnight. After exchange of the solvent with chloroform, a spatula's tip of K₂CO₃ was added and the mixture was left stirring for 4 hours at room temperature. After filtration, the product methyl-P[MeOx₂₆-b-BuOx₂₀-b-MeOx₂₈]-piperidine (598 mg, 0.083 mmol, 65% yield) was obtained as a colorless solid after precipitating the chloroform solution twice from cold diethylether.

### Example 2

### Preparation of Methyl-P [MeOx₂₇-b-BuOx₁₅-b-MeOx₂₇]-piperidine (LXRB15)

Using 24 mg MeOTf (0.146 mmol, 1 eq) as an initiator, MeOx (332.8 mg first block (3.91 mmol, 27 eq), 333.2 third block (3.91 mmol, 27 eq)) and 286.3 mg BuOx (2.25 mmol, 15 eq) and 80 μL of piperidine as terminating reagent, methyl-P[MeOx₂₇-b-BuOx₁₅-b-MeOX₂₇]-piperidine was prepared according to the general procedure described in Example 1.

### Example 3

### Paclitaxel 2 mg/mL

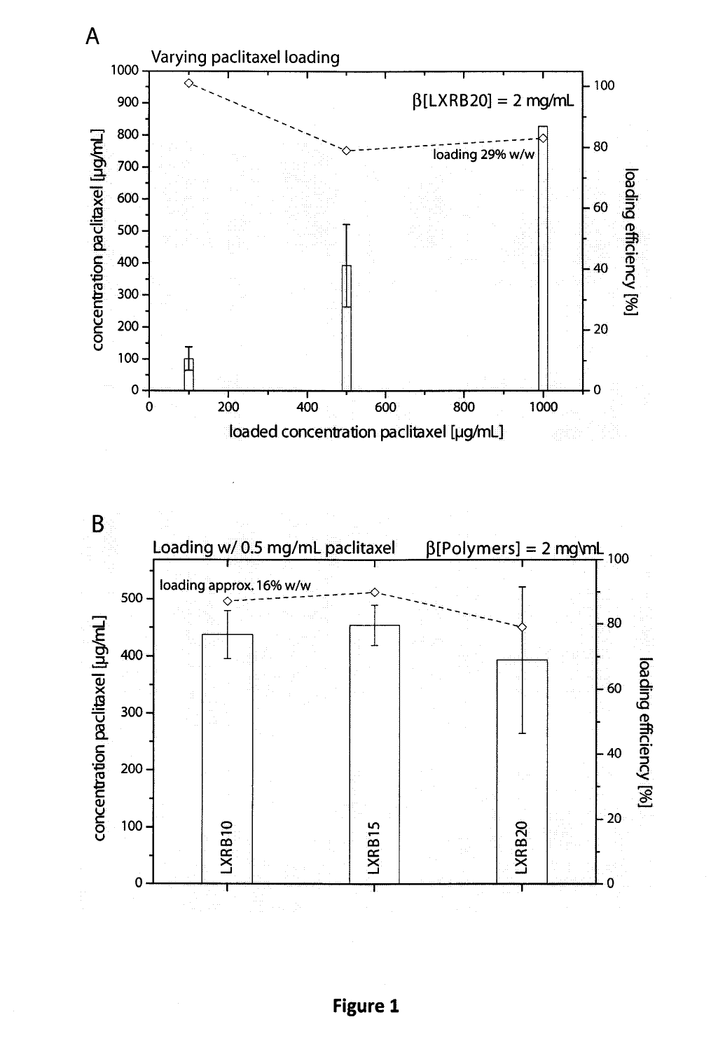

The enhanced solubilization of 2-butyl-2-oxazoline derived polymers is illustrated in this example. The polymers (400 μg) and paclitaxel (20, 100 and 200 μg, dissolved in acetonitrile, stock solution 5 mg/mL) were dissolved in 200 μL acetonitrile. The solvent was removed in a stream of air (or nitrogen or any other non-reactive gas) and the film was subjected to 0.2 mbar for at least 3 hours to remove residual solvent. Subsequently, 200 μL of buffer (aqueous solution, containing 122 mM NaCl, 25 mM Na₂CO₃, 10 mM HEPES, 10 mM glucose, 3 mM KCl, 1.4 mM CaCl₂ and 0.4 mM K₂HPO₄, pH=7.4) were added to obtain a final polymer concentration of 0.2 mg/mL (=2% (w/w)). The solution was filtered through syringe filters (0.45 μm pore size) and subjected to high performance liquid chromatography (HPLC) analysis. HPLC analysis was carried out under isocratic conditions using a Shimadzu system comprising a SCL-10A system controller, SIL-10A autoinjector, SPD-10AV UV detector and two LC-10 AT pumps. A Nucleosil® C18-5μ column (250 mm×4 mm) was used as the stationary phase and an acetonitrile/water mixture (55/45, v/v) was used as the mobile phase. Detection was performed at 220 nm. The amount of paclitaxel in the polymer solution was calculated using a calibration curve obtained using known amounts of paclitaxel dissolved in acetonitrile and analyzed accordingly. The results are shown in FIG. 1.

As seen in FIG. 1, the compositions were capable of solubilizing increasing amounts of paclitaxel. Even at a low polymer concentrations of 2 mg/mL, more than 0.8 mg/mL paclitaxel could be solubilized in aqueous solutions with these compositions, giving a loading capacity of approximately 30% (w/w). Surprisingly, the length of the hydrophobic block appears to have a limited effect (FIG. 1B). Decreasing the length of the hydrophobic block from 20 to 10 monomer units does not significantly diminish the drug loading capacity of the respective compositions.

### Example 4

### Paclitaxel 10 mg/mL Polymer

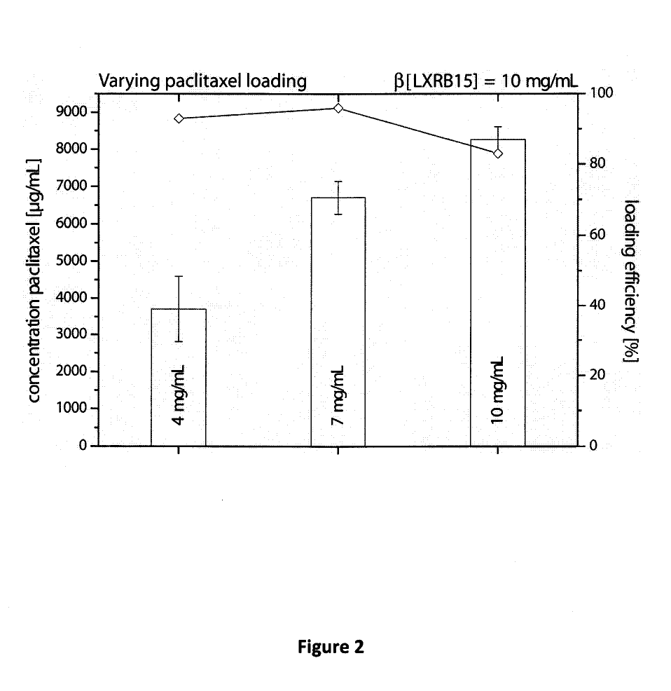

Following the procedure of Example 3, aqueous solutions of pharmaceutical composition comprising LXRB15 (10 mg/mL, 1% w/v) and various amounts of paclitaxel were prepared and analyzed subsequently. The results are presented in FIG. 2.

FIG. 2 shows the amount of paclitaxel solubilized in aqueous solutions within paclitaxel-LXRB15 compositions. Depending on the attempted drug loading, up to 8.3 mg/mL paclitaxel was found in aqueous solutions of compositions comprising 10 mg/mL LXRB15. This corresponds to a final drug loading of 45% (w/w) and a loading efficiency of 83%.

The size of the aggregates was determined using dynamic light scattering. For example, the Z-average size of the aggregates formed by the composition comprising 10 mg/mL LXRB15 and 3.7 mg/mL paclitaxel was found to be 20.7 nm with a very narrow size distribution (PDI=0.043). Similar values, ranging from 20-30 nm in diameter have been found for other compositions with also typically very narrow size distributions.

### Example 5

### Paclitaxel Freeze Drying

Polymer amphiphile solutions with solubilized paclitaxel where frozen to −80° C. and subsequently freeze dried. After taking the dry, colorless powders up with water to give clear solutions without any visible solid particles, they where subjected to centrifugation at 16,000×g for 15 minutes to sediment eventually present solids. Finally the solutions where subjected to HPLC analysis as described in Example 3. The results are presented in Table 1.

TABLE 1

| | Conc. | Conc. | Paclitaxel | Loading |
|---|---|---|---|---|
| Composition | Polymer | Paclitaxel | Loading | Efficiency |
| LXRB15 + | 10 mg/mL | 7.46 mg/mL | 43% | 75% |
| paclitaxel | | | | |
| LXRB15 + | 10 mg/mL | 6.62 mg/mL | 40% | 88% |
| paclitaxel | | | | |

This example shows clearly that the compositions of the present invention can be freeze dried, allowing prolonged storage as dry powders and easy reconstitution (e.g., by untrained personnel in a hospital setting), while retaining extraordinarily high drug loading.

### Example 6

### Cyclosporin A

To demonstrate the feasibility of cyclosporin A (CsA) containing compositions, 1 mg of LXRB15 was dissolved in 100 μL of acetonitrile. 50 μL of a 5 mg/mL cyclosporin A solution in ACN was added. Processing of the formulations was performed according to the procedure outlined above, using 200 μL of aqueous buffer. Isocratic HPLC analysis was performed at 70° C. using a mobile phase of 90% aqueous acetonitrile. The aqueous solution of the compositions was found to comprise 1.03 mg/mL CsA. Thus, drug loading was 17% (w/w) and loading efficiency was 82%. Under the same conditions, 8 μg/mL CsA was found to be solubilized in the aqueous buffer without amphiphilic block copolymer. Thus, compositions of the present invention can increase the solubility of cyclosporin A in a 0.5% (w/w) aqueous solution of the amphiphilic block copolymer LXRB15 at least 130 times.

The drug content of the composition was again analyzed after 3 days. While no change for the block copolymer cyclosporin A composition was found, the aqueous solution of cyclosporin A contained no detectable CsA. This shows that the compositions are of considerable stability and can be stored in aqueous solution for at least 3 days.

### Example 7

### Further Studies of Polymers

Table 2 provides the polymers used for the solubilization of paclitaxel, in accordance with the methods described hereinabove.

TABLE 2

| | | Molar |
|---|---|---|
| Sample | | mass* |
| Name | Polymer Composition* | [kg/mol] |
| LXRB10 | M[MeOx₂₆-b-BuOx₁₀-b-MeOx₂₆]Pid | 5.8 |
| LXRB15 | M[MeOx₂₆-b-BuOx₁₅-b-MeOx₂₆]Pid | 6.4 |
| LXRB20 | M[MeOx₂₆-b-BuOx₂₀-b-MeOx₂₆]Pid | 7.0 |
| LXR426 | B[BuOx₂₅-b-MeOx₅₃]BPip | 8.3 |
| LXR429 | T[BuOx₂₀-b-MeOx₁₀₀]BPip | 9.7 |
| LXR430T4 | B[MeOx₂₆-b-BuOx₁₅-b-MeOx₂₆]Pid | 6.8 |
| LXR434 | T[NonOx₈-b-MeOx₅₂]Pid | 5.0 |
| LXR438 | B[BuOx₁₅-b-MeOx₅₂]Pip | 6.8 |

*as determined by [M]₀/[I]₀;
M: methyltriflate initiated polymer;
B: 1,2-(N-methylbisoxazolinyliumtriflate) ethane initiated polymer;
T: tetrakistriflate pentaerithritol initiated polymer;
MeOx: 2-methyl-2-oxazoline;
BuOx: 2-butyl-2-oxazoline;
NonOx: 2-nonyl-2-oxazoline;
Pid: piperidine terminated polymer;
Pip: piperazine terminated polymer;
Bpip: N-Boc-piperazine terminated polymer

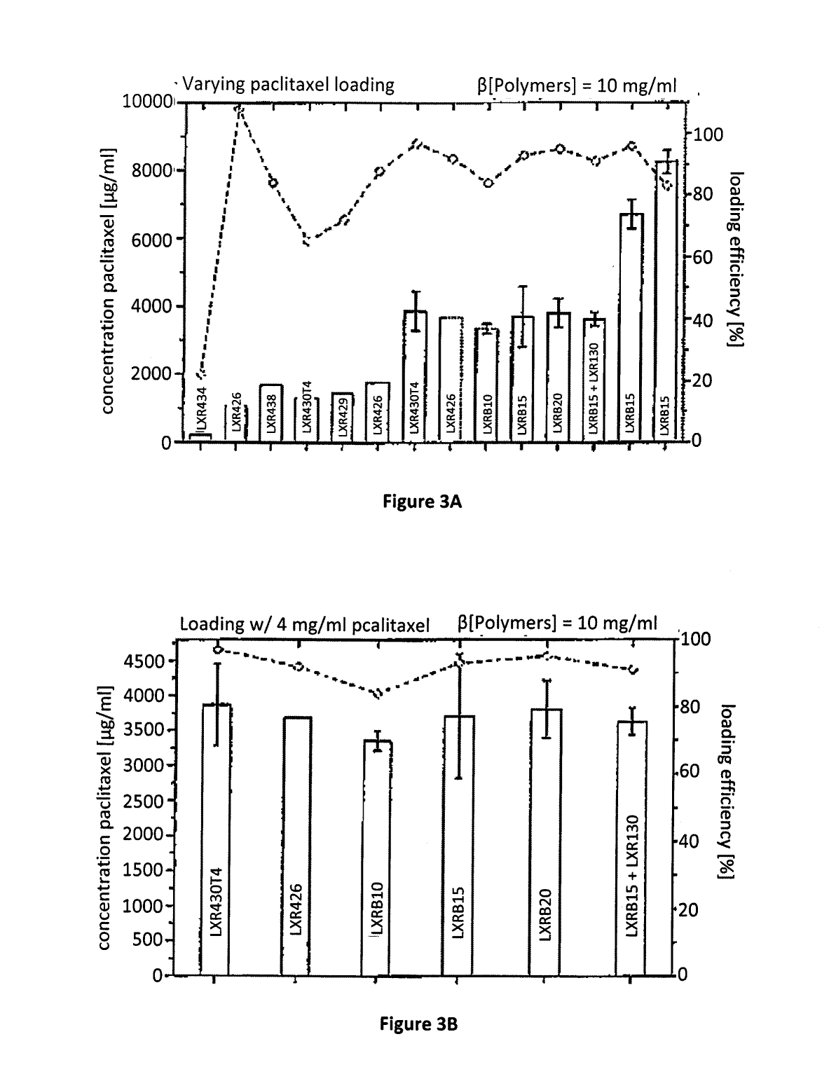

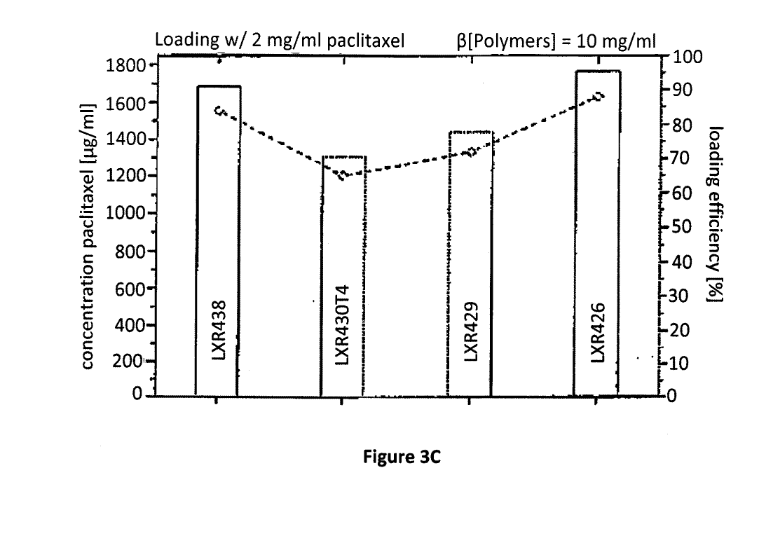

FIG. 3 demonstrates the solubilization of paclitaxel in micelles of various amphiphilic poly(2-oxazoline)s. The columns show the paclitaxel concentration in aqueous micelle solution as determined by HPLC. The line graph represents the loading efficiency ([paclitaxel] det/[paclitaxel]₀×100%). The polymer concentration in FIGS. 3A-3C is 10 mg/ml. FIG. 3A provides an overview of the solubilization power of various polymers at various paclitaxel loading concentrations. The first entry, which shows a very low loading efficiency, is a polymer which contains 2-nonyl-2-oxazoline instead of 2-butyl-2-oxazoliie as the hydrophobic monomer. FIGS. 3B and 3C show the solubilization of paclitaxel and loading efficiencies for various different polymers at loading concentrations of 4 and 2 mg/mL, respectively.

### Example 8

### Comparison to Cremophor EL®

To demonstrate the benefit of the present invention, the solubilization of compositions of the present invention was compared with the most commonly used, commercially available dispersant for paclitaxel, namely, a 50/50 (v/v) mixture of Cremophor EL® and dehydrated ethanol. In order to obtain a paclitaxel content of 4 mg/mL (a concentration needed to allow single bolus i.v. injection (100 μL) of a 20 mg/kg dose in mice), an aqueous solution containing 66% (v/v) of the commercially available paclitaxel/Cremophor EL® formulations would have to be prepared, containing 613 mg excipient per mL of solution. Using compositions of the present invention, a 4 mg/mL paclitaxel content can be achieved using as little as 5 mg/mL amphiphilic block copolymer or less, thereby decreasing the amount of excipient needed approximately 120 times.

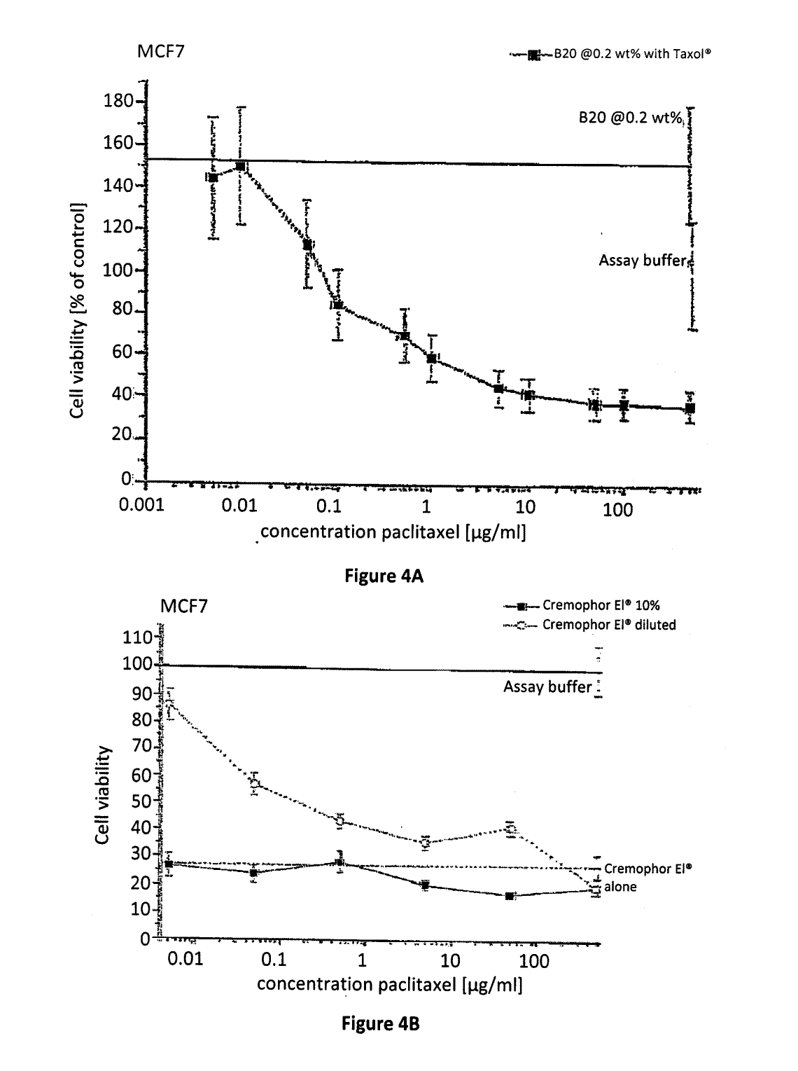

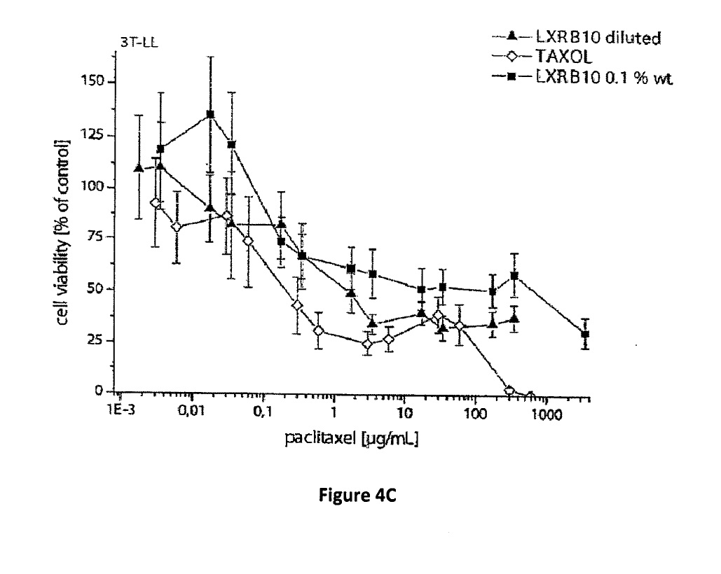

The toxicity of paclitaxel solubilized in LXRB20 was also compared to the toxicity of Cremophor EL®. As seen in FIGS. 4A and 4B, paclitaxel solubilized in LXRB20 has a toxicity comparable to paclitaxel solubilized in Cremophor EL® on the MCF-7 human breast cancer cell line. FIG. 4C demonstrates that paclitaxel solubilized in LXRB10, even when diluted, has a comparable IC₅₀ (approx. 0.1 μg/ml/l nM) to paclitaxel alone.

### Example 9

As stated herein, a majority of most potent drugs against serious diseases share a common flaw, which is a lack of water solubility. Thus, such drugs need to be formulated for parenteral administration. One prominent example in cancer chemotherapy is paclitaxel (PTX), a natural product of the bark of the pacific yew taxus brevifolia. It has a reported solubility in water of only 0.3 μg to 1 μg/mL, albeit depending on its crystallization state (Liggins et al. (1997) J. Pharm. Sci., 86:1458-1463; Lee et al. (2003) Pharm. Res., 20:1022-1030). Currently, two modi operandi of paclitaxel formulation are approved for human use. Typically, a mixture of Cremophor EL® (polyoxyethylated castor oil) and dehydrated ethanol is used to solubilize 6 mg/mL paclitaxel. However, serious formulation-evoked side effects have been reported (Pradis et al. (1998) Anticancer Res., 18:2711-2716; Gelderblom et al. (2001) Eur. J. Cancer, 37:1590-1598; Hennenfent et al. (2006) Ann. Oncol., 11:135-74), which make extensive premedication necessary. ABI-007 (Abraxane™, Abraxis Bioscience, Los Angeles, Calif.), a nanoparticulate (size approx. 130 nm) albumin-paclitaxel formulation can overcome some of the problems encountered with Taxol® and is currently approved for treatment of relapsed breast cancer. It allows injections of paclitaxel at a concentration of 5 mg/mL. However, it still contains 90% wt. of carrier and only 10% wt. of drug. Herein, novel nanoformulations are reported which have unprecedentedly high loading capacity and contain at least 40% wt. of paclitaxel incorporated in non-toxic, small (20 nm diameter) poly(2-oxazoline)-based polymeric micelles. The formulations are very simple to prepare, stable, and can be lyophilized and readily re-dispersed without cryoprotectants. They are shown to deliver at least 8 mg/mL of drug in the active form to treat cancer.

Poly(2-oxazoline)s have recently attracted increasing attention for biomedical applications. Of particular interest are hydrophilic poly(2-methyl-2-oxazoline) (PMeOx) and poly(2-ethyl-2-oxazoline) (PEtOx) as they exhibit stealth (Zalipsky et al. (1996) J. Pharm. Sci., 85:133-137; Woodle et al. (1994) Bioconjugate Chem., 5:494-496) and protein repellent (Komadi et al. (2008) Langmuir 24:613-616) effects similar to polyethylene glycol, arguably the most commonly used polymer for injectable drug delivery systems. In contrast to polyalkylene glycols the poly(2-oxazoline)s hydrophobicity can be gradually fine-tuned in a very broad range.

### Materials and Methods

### Preparation of Polymer Amphiphiles

The polymerizations and work-up procedures were carried out according to the procedure described previously (Luxenhofer et al. (2006) Macromolecules, 39:3509-3516).

As an example, the preparation of methyl-P[MeOx₂₇-b-BuOx₁₂-b-MeOx₂₇]-piperidine (P1) was performed as follows. Under dry and inert conditions 32.2 mg (0.2 mmol, 1 eq) of methyl trifluoromethylsulfonate (methyl triflate, MeOTf) and 440 mg (5.17 mmol, 26 eq) of 2-methyl-2-oxazoline (MeOx) were dissolved in 3 mL dry acetonitrile at room temperature. The mixture was subjected to microwave irradiation (150 W maximum, 130° C.) for 15 minutes. After cooling to room temperature, the monomer for the second block, 2-butyl-2-oxazoline (256 mg, 2.01 mmol, 10 eq) was added and the mixture was irradiated the same way as for the first block. The procedure was repeated for the third block with 442 mg (5.19 mmol, 26 eq). Finally, P1 was terminated by addition of 0.1 mL piperidine (1.01 mmol, 5 eq) at room temperature. After stirring over night, an excess of K₂CO₃ was added and the mixture was allowed to stir for several hours. The mixture was concentrated after filtration and added to 3 mL of chloroform. After precipitation from cold diethyl ether (approx. 10 times the amount of polymer solution) the product was obtained by centrifugation. The precipitation was performed in triplicate and the polymer was obtained as a colorless powder (792 mg, 67%, Mₜₕ, =5.8 kg/mol) after lyophilization from water. GPC (DMAc): Mₙ=8.5 kg/mol (PDI 1.21); ¹H-NMR (CDCl₃, 298 K): δ=3.45 (br, 255H, (N—CH₂CH₂)); 3.04/2.95 (m, 3H, N—CH₃^{Ini}); 2.43-1.86 (m, 212H, CO—CH₃, CO—CH₂, CH₂^{Pid}); 1.56 (br, 29H, CH₂—CH₂—CH₂—); 1.32 (br, 28H, —CH₂—CH₃); 0.91 ppm (br, 37H, —CH₃^{butyl}), Mₙ=6.2 kg/mol (MeOx₂₇-b-BuOx₁₂-b-MeOx₂₇).

Preparation of Methyl-P[MeOx₃₇-b-BuOx₂₃-b-MeOx₃₇]-piperidine (P2)

P2 was obtained in a similar manner using 24 mg MeOTf (0.146 mmol, 1 eq), 333 mg MeOx (3.91 mmol, 27 eq, 1st block), 286 mg BuOx (2.25 mmol, 15 eq, 2^{nd} block) and 333 mg MeOx (3.91 mmol, 27 eq, 3^{rd} block) and 80 μL of piperidine as terminating reagent. The product was obtained as a colorless solid (795 mg, 83%, Mₜₕ=6.6 kg/mol). GPC (DMAc): Mₙ=10.4 kg/mol (PDI 1.18); ¹H-NMR (CDCl₃, 298 K): δ=3.44 (br, 360H, (N—CH₂CH₂)); 3.03/2.94 (m, 3H, N—CH₃^{Ini}); 2.33-1.9 (m, 279H, CO—CH₃, CO—CH₂, CH₂^{Pid}); 1.55 (br, 47H, CH₂—CH₂—CH₂—); 1.32 (br, 45H, —CH₂—CH₃); 0.91 ppm (br, 68H, —CH₃^{butyl}), Mₙ=9.3 kg/mol (MeOx₃₇-b-BuOx₂₃-b-MeOx37).

Preparation of Methyl-P[MeOx₃₆-b-BuOx₃₀-b-MeOx₃₆]-piperidine (P3)

P3 was prepared accordingly using 24.7 mg methyltriflate (0.150 mmol, 1 eq) and 334 mg 2-methyl-2-oxazoline (3.9 mmol, 26 eq, 1^{st} block). An aliquot of 136 mg (5% w/w) of the reaction mixture where removed for analysis of the first block with NMR and GPC. The same procedure was performed after the second block (364.4 mg BuOx; 2.87 mmol, 20 eq, 10% w/w analyzed). Block three (306.9 mg MeOx; 3.6 mmol, 28 eq) was added, the polymerization was terminated using 80 μL piperidine and the product was obtained as a colorless solid (598 mg, 65%, Mₜₕ=6.6 kg/mol). GPC (DMAc): Mₙ=9.9 kg/mol (PDI 1.23); ¹H-NMR (CDCl₃, 298 K): δ=3.45 (br, 405H, (NCH₂CH₂)); 3.03/2.95 (m, 3H, N—CH₃^{Ini}); 2.43-1.86 (m, 329H, CO—CH₃, CO—CH₂, CH₂^{Pid}); 1.57 (br, 63H, CH₂—CH₂—CH₂—); 1.32 (br, 60H, —CH₂—CH₃); 0.91 ppm (br, 88H, CH₃^{butyl}), Mₙ=10.0 kg/mol (MeOX36-b-BuOx₃₀-b-MeOX₃6).

Preparation of Methyl-P[EtOx₅₀-b-BuOx₁₉]-piperazine (P4)

P4 was prepared accordingly from 10 mg MeOTf (61 μmol, 1 eq), 321 mg 2-ethyl-2-oxazoline (3.24 mmol, 53 eq, 1^{st} block) and 157 mg BuOx (1.23 mmol, 20 eq, 2^{nd} block), using 150 mg piperazine as a terminating reagent. For precipitation, a solvent mixture of cyclohexane and diethylether (50/50, v/v) was used. The product was obtained as a colorless solid (yield 0.36 g, 77%, Mₜₕ=7.8 kg/mol). GPC (DMAc): Mₙ=11.5 kg/mol (PDI 1.09); ¹H-NMR (CDCl₃, 298 K): δ=3.45 (br, 276H, (NCH₂CH₂)); 3.04/2.95 (m, 3H, N—CH₃^{Ini}); 2.5-2.2 (m, 144H, CO—CH₂—CH₃, CO—CH₂, CH₂^{Pid}); 1.58 (br, 37H, CH₂—CH₂—CH₂—); 1.34 (br, 41H, —CH₂—CH₃); 1.11 (br, 151H, CO—CH₂—CH₃); 0.91 ppm (br, 56H, —CH₃^{butyl}), Mₙ=7.5 kg/mol (EtOx₅₀-b-BuOx₁₉).

Preparation of Methyl —P[MeOx₄₂-b-BuOx₁₈-b-MeOX₄₂]-piperazine (P5)

P5 was prepared accordingly from 14 mg MeOTf (85 μmol, 1 eq), 190 mg MeOx (2.2 mmol, 26 eq, 1^{st} block), 236 mg BuOx (1.86 mmol, 22 eq, 2^{nd} block) and 192 mg MeOx (2.3 mmol, 27 eq, 3^{rd} block) using 200 mg piperazine as a terminating reagent. The product was obtained as a colorless solid (0.47 g, 69%, Mₜₕ=8.0 kg/mol) GPC (DMAc): Mₙ=14.7 kg/mol (PDI 1.22); ¹H-NMR (CDCl₃, 298 K): δ=3.45 (br, 408H, (NCH₂CH₂)); 3.04/2.95 (m, 3H, N—CH₃^{Ini}); 2.4-2.0 (m, 307H, CO—CH₃, CO—CH₂, CH₂^{Pid}); 1.56 (br, 37H, CH₂—CH₂—CH₂—); 1.33 (br, 37H, —CH₂—CH₃); 0.91 ppm (br, 53H, —CH₃^{butyl}), Mₙ=9.5 kg/mol (MeOx₄₂-b-BuOx₁₈-b-MeOx₄₂).

TABLE 3
Analytical data and composition of amphiphilic
block copolymers used.

| | Polymer | Mₙ^{a} | Mₙ^{b} | | Yield |
|---|---|---|---|---|---|
| | Composition | [kg/mol] | [kg/mol] | PDI^{b} | [%] |
| P1 | MeOx₂₇-b-BuOx₁₂-b- | 6.2 | 8.5 | 1.21 | 67 |
| | MeOx₂₇ | | | | |
| P2 | MeOx₃₇-b-BuOx₂₃-b- | 9.3 | 10.4 | 1.18 | 83 |
| | MeOx₃₇ | | | | |
| P3 | MeOx₃₆-b-BuOx₃₀-b- | 10.0 | 9.9 | 1.23 | 65 |
| | MeOx₃₆ | | | | |
| P4 | EtOx₅₀-b-BuOx₁₉ | 7.2 | 11.5 | 1.09 | 77 |

^{a}as determined by endgroup analysis from 1H-NMR spectroscopy.
^{b}as determined by gel permeation chromatography.

### Attachment of Fluorophore (At to 425)

Labeling of piperazine terminated polymers P4 and P5 was performed in anhydrous dimethylformaide (DMF) and diisopropylethylamine (DIPEA) with 1.2 eq of reactive dye (Atto425-NHS ester, Sigma-Aldrich, St. Louis, Mo.) per eq of polymer. Reaction was stirred for 3 days at room temperature in the dark and diluted with methanol. Remaining free dye was removed by gel filtration (Sephadex™ LH20) in methanol which was performed in triplicate.

Critical Micelle Concentration (cmc) Measurement; Pyrene Assay:

The critical micelle concentration (cmc) was determined using described method (Kabanov et al. (1995) Macromolecules, 28:2303-2314; Colombani et al. (2007) Macromolecules, 40:4338-4350). In short, a pyrene solution in acetone (2.5 mM) was added to vials and the solvent was allowed to evaporate. Polymer solutions at appropriate concentrations in assay buffer were added to the vials so that a final concentration of 5×10⁻⁷M of pyrene was obtained. The solutions were incubated at 25° C. (22 hours) and the pyrene fluorescence spectrum were recorded using a Fluorolog®3 (HORIBAJobinYvon) λₑₓ=333 nm, λₑₘ=360-400 nm, slidwidth(ex)=slidwidth(em)=1 nm, step width 0.5 nm. Typically, five spectra of each data point were averaged (integration time 0.1 seconds, if necessary 10 spectra with 0.2 seconds integration), the cmc is assumed where a steep increase in fluorescence intensity is observed. Furthermore, the fluorescence intensity of the I₁ band was compared to the intensity of I₃ band which gives an estimate of the polarity of the environment of the pyrene probe.

### Drug Solubilization Studies

Drug-polymer solutions were prepared using the thin film method. Appropriate amounts of polymer and paclitaxel (stock solution 5 mg/mL) were solubilized in minimum amounts of acetonitrile (ACN). The solvent was removed in a stream of air under mild warming and the films were subjected to 0.2 mbar for at least 3 hours to remove residual solvent. Subsequently 200 μL of assay buffer (aqueous solution, containing 122 mM NaCl, 25 mM Na₂CO₃, 10 mM HEPES, 10 mM glucose, 3 mM KCl, 1.4 mM CaCl₂, and 0.4 mM K₂HPO₄, pH=7.4) were added to obtain final polymer concentration as mentioned in the main text. At higher paclitaxel concentration solubilization was facilitated by incubation of the solutions for 50-60° C. for typically 5-10 minutes. The clear solutions were filtered through HPLC syringe filters (0.45 μm pore size) and subjected to HPLC analysis. In the eye of future application in vivo, it is also noteworthy that substitution of the relatively toxic ACN with the more benign EtOH as a common solvent before film formation did not diminish loading efficiencies.

### HPLC Analysis of Drug Solubilization

HPLC analysis was carried out under isocratic conditions using a Shimadzu system comprising a SCL-10A system controller, SIL-10A autoinjector, SPD-10AV UV detector and two LC-10 AT pumps. As stationary phase a Nucleosil® C18-5μ column was used (250 mm×4 mm), as a mobile phase an acetonitrile/water mixture (55/45, v/v) was applied. Detection was performed at 220 nm. The amount of paclitaxel in the polymer solution was calculated using a calibration curve obtained with known amounts of paclitaxel dissolved in acetonitrile and analyzed accordingly.

### NMR

For NMR analysis, paclitaxel containing polymer thin films were dissolved in the respective deuterated solvents (acetonitrile-d₃, chloroform-d₁ or 20% (v/v) D₂O in H₂O).

### Dynamic Light Scattering

Dynamic light scattering was performed using a Zetasizer Nano-ZS (Malvern Instruments Inc., Southborough, Mass.) at room temperature.

### Cell Culture

MCF7-ADR cells were derived from human breast carcinoma cell line, MCF7 (ATCC HT-B22), by selection with Doxorubicin.

### MTT Assay

MCF7/ADR were seeded in 96 well plates (10⁴ cells per well) and were allowed to reattach for 24 hours. Treatment solutions were prepared from a 1 mg/mL polymer stock solution in assay buffer (containing 122 mM NaCl, 25 mM NaHCO₃, 10 mM glucose, 10 mM HEPES, 3 mM KCl, 1.2 mM MgSO₄, 1.4 mM CaCl₂, and 0.4 mM K₂HPO₄, pH 7.4) by appropriate dilution with media (Dulbecco's Modified Eagle's Medium (DMEM), supplemented with 10% fetal bovine serum (FBS), 25 mM HEPES and penicillin/streptomycin). The cells were incubated for 48 hours with 200 μL of treatment solution. After discarding the treatment solution, cells were washed thrice with PBS. FBS-free DMEM (100 μl/well) as well as 25 μL of a 5 mg/mL solution of 3-(4,5-dimethylthiazol-2-yl)-2,5-diphenyltetrazolium bromide (MTT, Invitrogen, Eugene, Oreg.) in PBS were added and the cells incubated at 37° C. for 2 hours. The media was discarded subsequently and replaced with 100 μL of solvent (25% v/v DMF, 20% w/v SDS in H₂O). The purple formazan product was allowed to dissolve over night and the absorbance at 570 nm was obtained using a plate reader (SpectraMax® M5, Molecular Devices). Positive control were cells treated with media alone, negative control were wells without cells. Each concentration was repeated in four wells, results are expressed as mean±SEM.

### Flow Cytometry

For the analysis of cellular uptake by flow cytometry, MCF7/ADR cells were plated in 24 well plates (7.5×10⁴ per well) two days prior to the experiment. Cells were treated with 200 μL of polymer solutions in FBS free media. In the case of experiment performed at 4° C., the cells were washed 3 times with ice cold PBS and incubated with ice-cold polymer solution. Cells were incubated for 60 minutes or the indicated time at 37° C./5% CO₂ or 4° C., washed subsequently thrice with ice-cold PBS, trypsinized and centrifuged. The cell pellet was resuspended in 400 μL PBS with 1% bovine serum albumin, split in two aliquots and analyzed using flow cytometry. Each data point was performed in triplicate. The mean fluorescence intensity was determined using a BD Biosciences LSRII digital flow cytometer operating under FACSDiVa® software version 6.1 (San Jose, Calif.). Excitation was provided by a 25 mW Coherent VioFlame™ PLUS violet laser (405 nm), and emission collected through a 450/50 bandpass filter. Approximately 10,000 digital list mode events were collected and the data gated on forward and side scatter parameters to exclude debris and dead cells. Control cells without labeled polymers were used as the negative control for autofluorescence. Data analysis was performed using DiVa® software.

### Confocal Fluorescence Microscopy

For live cell confocal microscopy (Carl Zeiss LSM 510 Meta, Peabody, Mass.) MCF7/ADR cells (4×10⁴) were plated in Lab-Tek Chambered Cover Glasses dishes (Fischer Scientific, Waltham, Mass.) and after two days (37° C., 5% CO₂) were exposed for 60 minutes to Atto-425 labeled polymer solutions in FBS free media. Subsequently, cells were washed (3×PBS) and kept in complete media for imaging using the confocal microscope. Alternatively, the cells were fixed with 4% paraformaldehyde solution for 10 minutes at room temperature, the PFA was substituted with PBS and the cells were kept at 4° C. in the dark until confocal microscopy was performed.

### Results

Notably, the most hydrophobic poly(2-oxazoline)s contain in each repeating unit a highly polar amide motif in the backbone, which makes these compounds nonionic polysoaps. By combining different poly(2-oxazoline)s in block copolymer structures, a special type of polymeric surfactants was produced with amphiphilicity embedded both in the block copolymer architecture and in every repeating unit of each block. Specifically, four well-defined ABA-type triblock copolymers (P1-P3) and one diblock copolymer (P4) of molar masses ca. 8 to 10 kg/mol and low polydispersities (PDI=1.09-1.23) were synthesized by living cationic ring opening polymerization. The hydrophilic blocks (A) consisted of 50 to 80 units of PMeOx (P1-P3) or PEtOx (P4), and the hydrophobic block (B) consisted of 10 to 22 units of 2-butyl-2-oxazoline (PBuOx) (Table 3). All these polymers readily dissolve in water at room temperature at concentrations of up to 15-30 wt. %.

The homologue series of poly(2-alkyl-2-oxazoline)s share a polar amide motif and display a gradually increasing hydrophobicity as the alkyl side chains increase in length. The series starts from highly hydrophilic poly(2-methyl-2-oxazoline), followed by slightly amphiphilic thermo-responsive poly(2-ethyl-2-oxazoline), then by more hydrophobic poly(2-isopropyl-2-oxazoline) and poly(2-propyl-2-oxazoline) and finally, by poly(2-butyl-2-oxazoline), which shows no marked aqueous solubility. The lower critical solution temperatures (LCST) depend on the molecular mass and the polymer structure (Huber et al. (2008) Colloid Polym. Sci., 286:395-402). LCSTs for the polymers are ˜70° C. for poly(2-ethyl-2-oxazoline), ˜40° C. for poly(2-isopropyl-2-oxazoline), and ˜25° C. for poly(2-propyl-2-oxazoline).

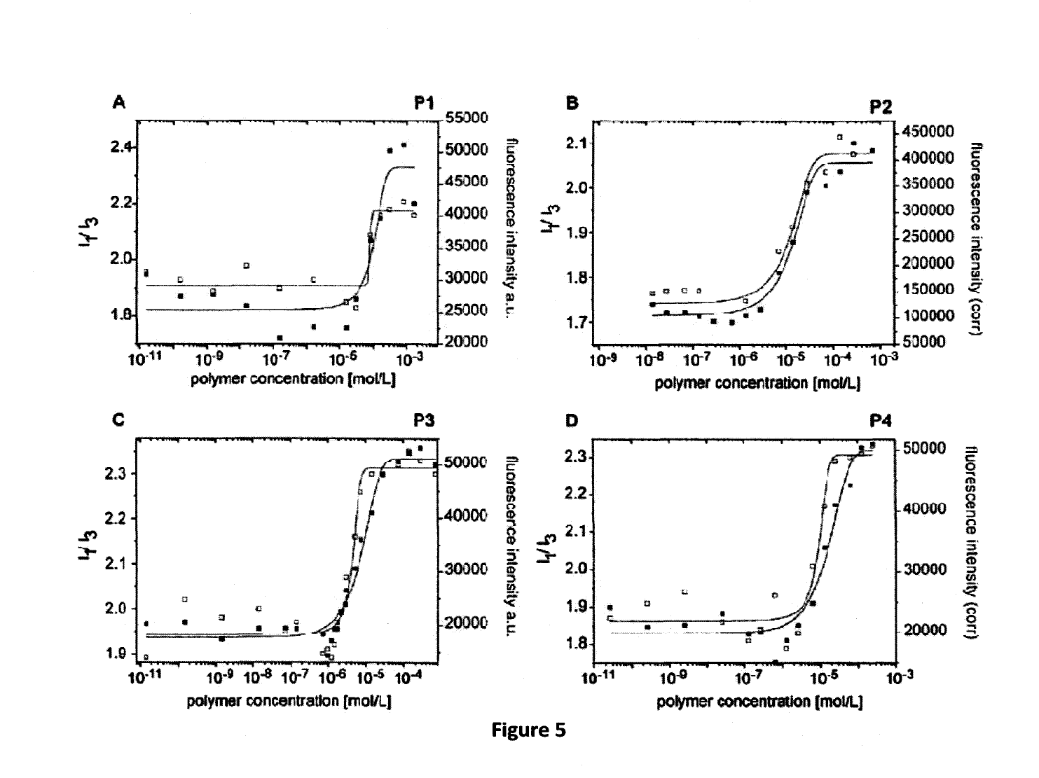

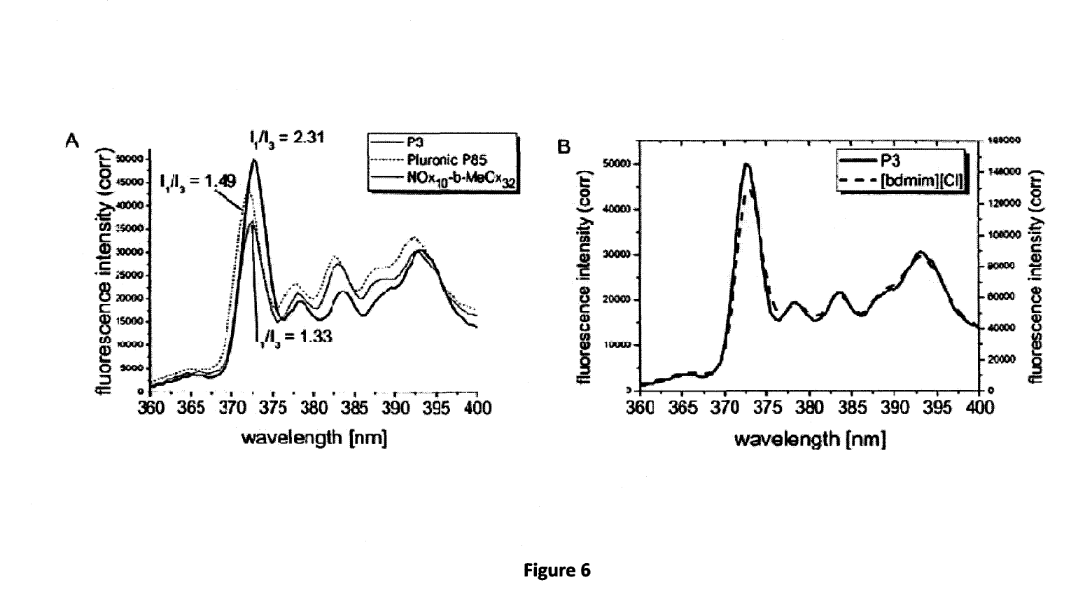

In order to prove that polymers P1-P4 self-assemble in polymeric micelles in aqueous solutions, pyrene was used as a highly hydrophobic fluorescence probe. The onset of increasing pyrene fluorescence intensity is typically observed as the polymer concentration reaches the critical micelle concentration (cmc) (Colombani et al. (2007) Macromolecules 40:4338-4350). Cmc's for polymers P1-P4 were found to be 100 mg/L (15 μM), 20 mg/L (2.7 μM), 7 mg/L (1 μM), and 6 mg/L (0.7 μM), respectively (FIG. 5A-5D). These very low cmc values are desirable when a parenteral application is considered, as any systemically administered polymer solution will be diluted rapidly by 100 to 1000 times. The ratio of I₁ and I₃ bands in the fluorescence emission spectrum of pyrene was used to test polarity of the environment of the pyrene probe. Indeed, the fine structure of the pyrene fluorescence spectra is known to correlate well with the permanent dipolar moment of the environment (typically solvent), while it correlates only poorly with the permittivity of the medium (Kalyanasundaram et al. (1977) J. Am. Chem. Soc., 99:2039-2044). When pyrene is in an aqueous or similarly polar environment, the I₁/I₃ ratio is found between 1.6 and 1.9, although it has been shown that the ratio is influenced both by environmental and instrumental conditions (Street et al. (1986) Analyst 111:1197-1201). When polymer aggregates are formed, a less polar environment is usually available for pyrene into which it is partitioned. As a result, the I₁/I₃ ratio usually decreases concomitantly with the increasing overall fluorescence intensity. Quite surprisingly, the opposite was observed. As the fluorescence intensity increased, the I₁/I₃ ratio also increased up to 2.35 (FIG. 6A). Moreover, the I₁/I₃ ratio increased as the size of “hydrophobic” BuOx block increased. This phenomenon is unique for polymeric micelles, or for any other media. It indicates that, as aggregates of P1-P4 form, the pyrene probe is translocated into an amphipolar environment, which is sufficiently hydrophobic to solubilize pyrene yet, more polar than water. Based on the I₁/I₃ ratio this environment is similar to a polar solvent, dimethylsulfoxide, or ionic liquid, 1-butyl-2,3-dimethylimidazolium chloride (FIG. 6B), rather than nonopolar solvent, hexane, or regular polymeric micelles of Pluronic® P85 (FIG. 6A). Such an environment is probably heterogeneous on the very small scale and is formed due to intrinsic amphiphilicity in every repeating unit of BuOx blocks of poly(2-oxazoline)s. Therefore, pyrene entraps in the hydrophobic domains formed by butyl moieties yet still comes in contact with the polar amide motifs. Consequently, replacement of butyl for 2-nonyl-2-oxazoline (NOx) in the core forming block of NOx₁₀-b-MeOx₃₂ completely reverses the I₁/I₃ ratio (FIG. 6A), presumably because now pyrene can be completely immersed in a hydrophobic domain formed by the bulky nonyl moieties. In contrast, while the butyl side chains lead to hydrophobic compartments, the polymer backbone remains hydrated due to the presence of the polar amide motif in every repeating unit, creating unique amphipolar environment for the solubilized molecules.

As stated above, observed I₁/I₃ ratios of pyrene fluorescence signals vary based on solvents and polymeric micelles. By way of example, hexanes yield an I₁/I₃ ratios of about 0.6. For polymeric micelles, values varying between 0.8 up to 1.5 are typically observed (e.g., Pluronic® block copolymers from about 1.2-1.5). Only few solvents yield ratios that are around or slightly above water (about 1.6-1.9), including dimethylsulfoxide (about 1.9-2.05), acetonitrile and in some cases, ionic liquids (about 1.8-2.1). 2-butyl-2-oxazoline based polymer amphiphiles were found to give much higher ratios than observed in water, indicating an amphipolar environment present in the micelle. 2-nonyl-2-oxazoline based polymer amphiphiles exhibited a ratio from about 1.2-1.4.

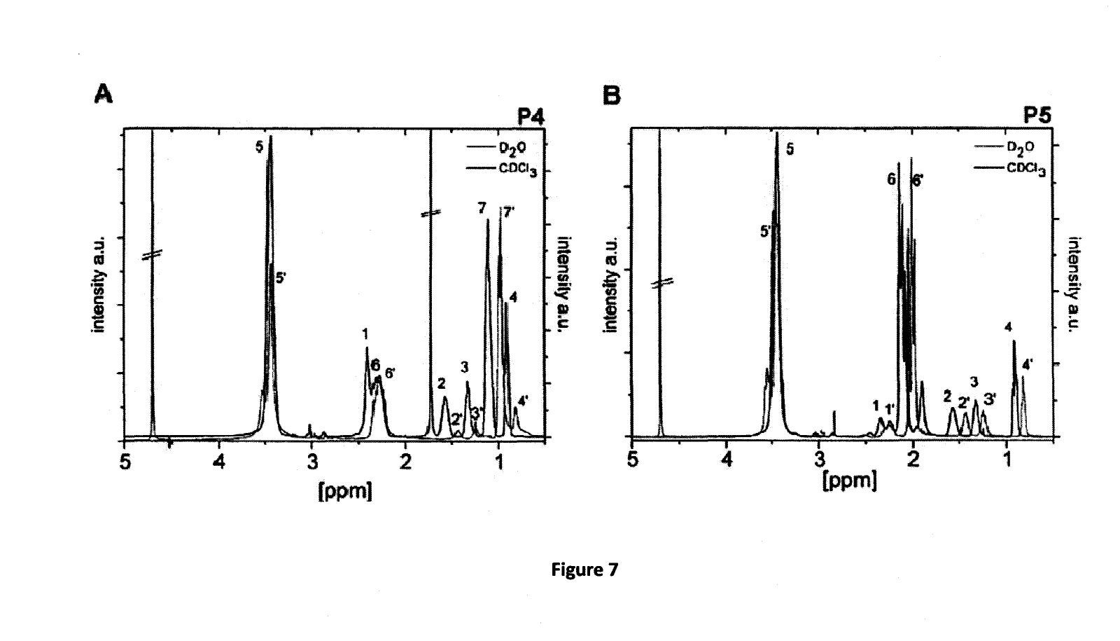

One should expect that the P1-P4 aggregates are highly hydrated due to the presence of the polar amide motif in the repeating units of poly(2-oxazoline)s. This was corroborated by the results of an ¹H-NMR study (FIG. 7). Clearly, when spectra of polymers are obtained under conditions when aggregates are present, the signals of the butyl side chains are markedly attenuated (signals 1-4 vs. 1′-4′; FIGS. 7A and 7B) compared to the corresponding signals of the hydrophilic blocks (signal 6/7 vs. 6′/7′). The signal originating from the polymer main chain (signal 5 and 5′, present in both hydrophilic and hydrophobic blocks), however, appears to be subject to less pronounced attenuation. These results indicate that the side chains of BuOx blocks segregate in domains with restricted solvent access. However, the fact that the signals remain well observable suggests that the “hydrophobic” part of the micelle is in fact well hydrated.

Surprisingly, these aggregates exhibited remarkable capability for solubilization of paclitaxel. To prepare drug loaded polymeric micelles a thin-film dissolution method was used. Poly(2-oxazoline)s are readily soluble in a wide range of organic solvents, including ethanol, dimethylsulfoxide, chloroform, acetonitrile and others, which greatly facilitates their formulation with water-insoluble drugs. Solutions of polymers and paclitaxel were simply combined in acetonitrile or ethanol and then the solvent was removed under a stream of air and vacuum. Upon addition of water the polymer-drug film dissolved rapidly and completely, if the concentration of paclitaxel did not exceed 4 mg/mL. At higher concentrations mild heating (<60° C.) was used to facilitate the process for P1-P3. For P5, an LCST-like behavior was observed around 50° C.

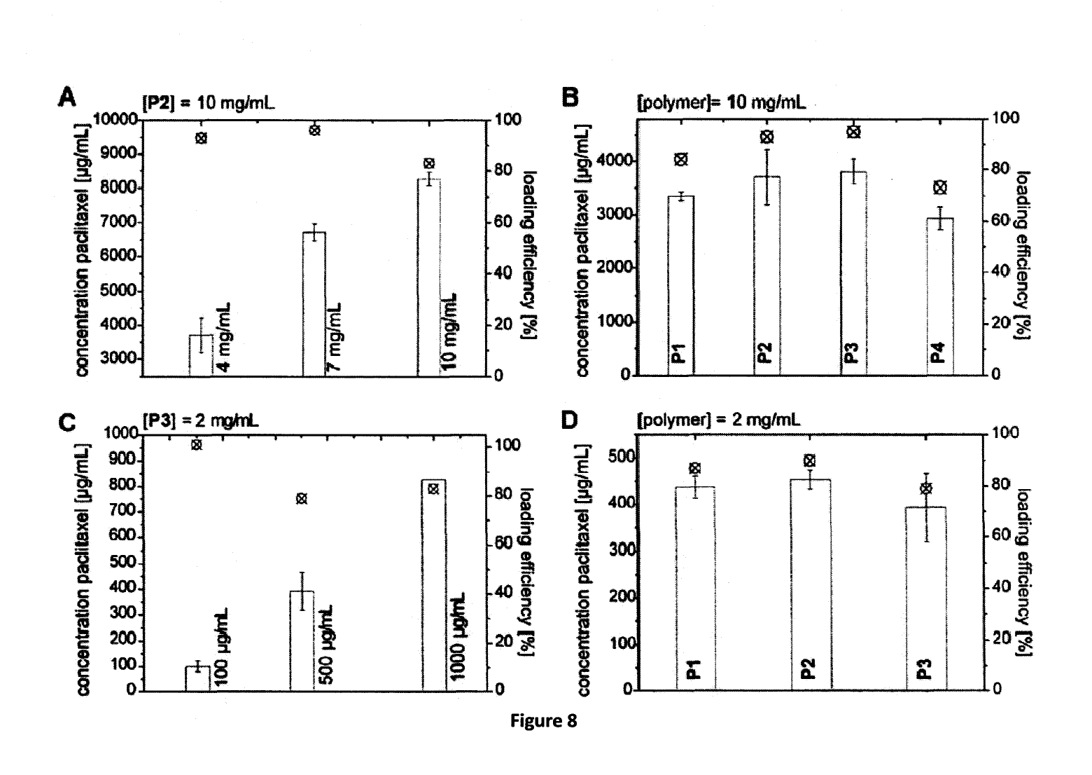

Initially, it was attempted to solubilize 4, 7 and 10 mg/mL paclitaxel with 10 mg/mL P2. Up to concentrations of 7 mg/mL paclitaxel, clear solutions were obtained after mild heating for a short time. Under these conditions the solubilization of paclitaxel was complete as confirmed by high performance liquid chromatography (HPLC) (FIG. 8A). Only at 10 mg/mL paclitaxel some clear crystals remained undissolved even after 30 minutes heating at 60° C. However, an extraordinary solubilization of paclitaxel of 8.2 mg/mL was still obtained, indicating that the resulting formulation consists of at least 40% wt. paclitaxel. Similar results were obtained with the other polymers including P1, having only 12 units in the BuOx block (FIG. 8B). Even at polymer concentrations as low as 2 mg/mL, excellent loading efficiencies and total drug loading of almost 30% wt. were obtained (FIGS. 8C and 8D). Notably, upon dilution of the drug-polymer solutions with acetonitrile (ACN) for subsequent HPLC analysis, the dissolved paclitaxel instantaneously precipitated at concentrations exceeding 1 mg/mL. This is a simple but convincing proof that the paclitaxel is indeed dissolved in polymer micelles which disintegrate upon addition of small amounts of ACN. However, upon appropriate dilution with water, the solutions remained clear and where analyzed after passing through HPLC-syringe filters. As compared to Cremophor EL® and Abraxane™, the poly(2-oxazoline) block copolymers can reduce the amount of excipient needed to solubilize paclitaxel by approx. 100 and 9 times, respectively.

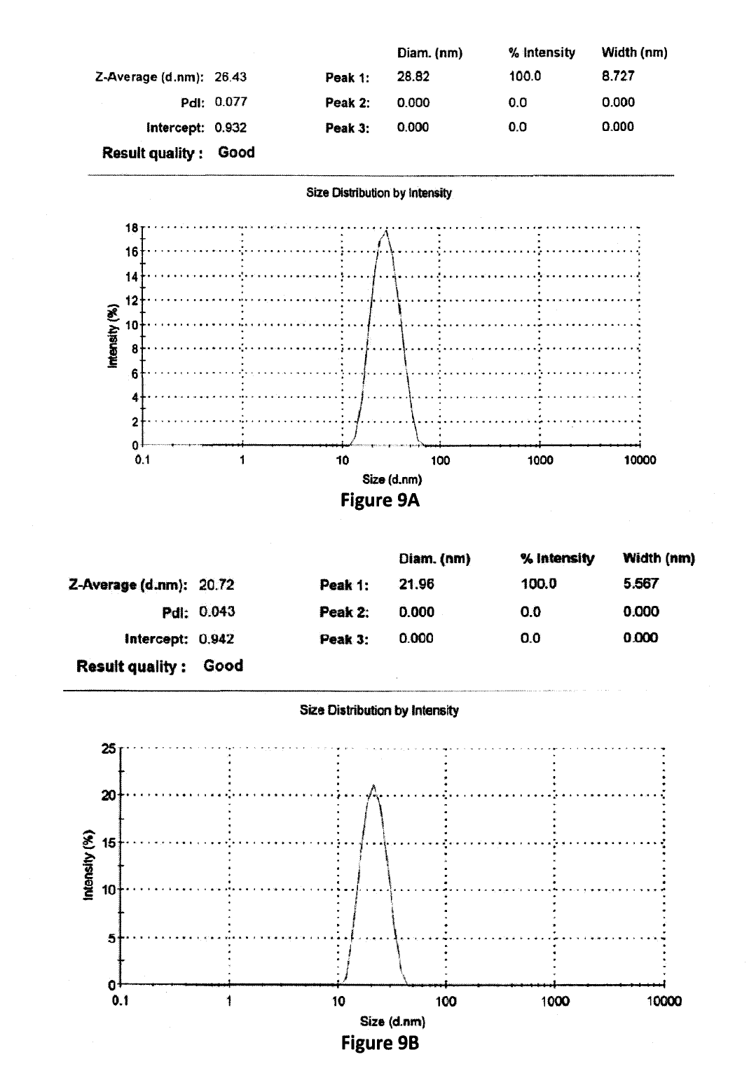

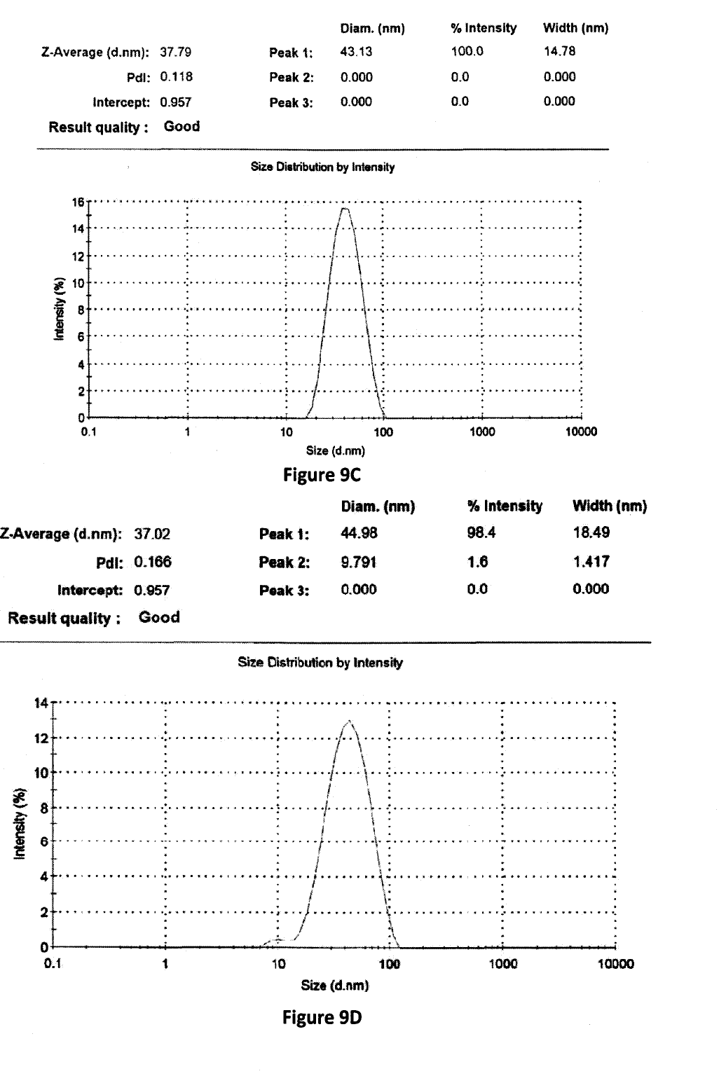

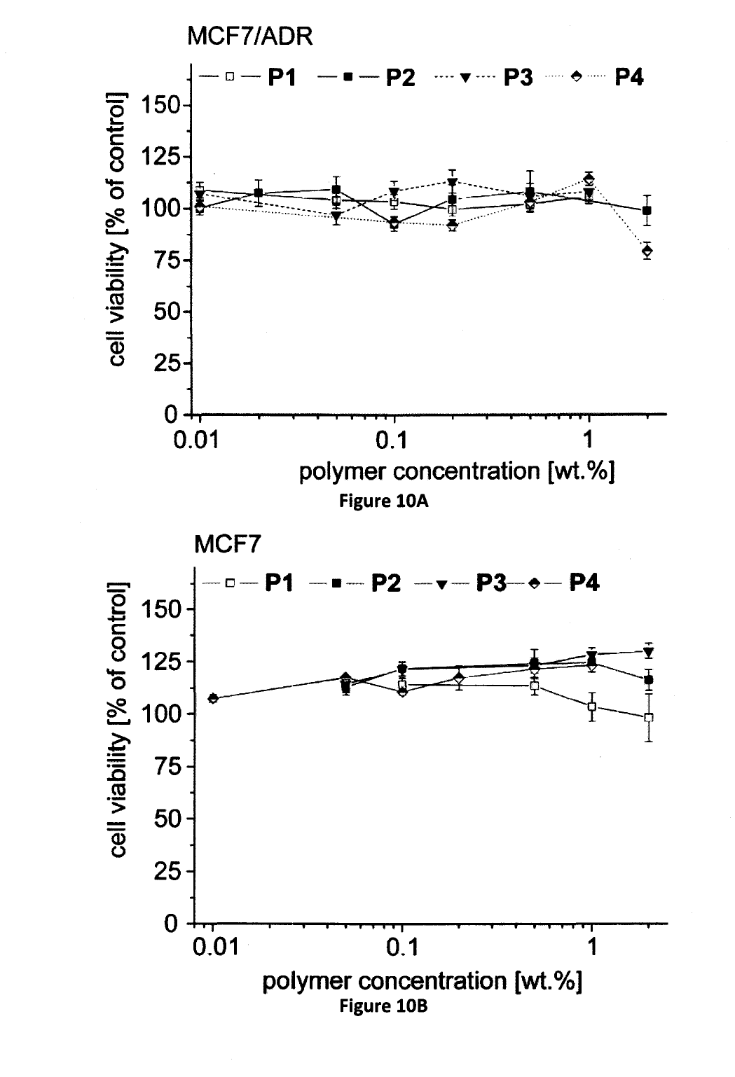

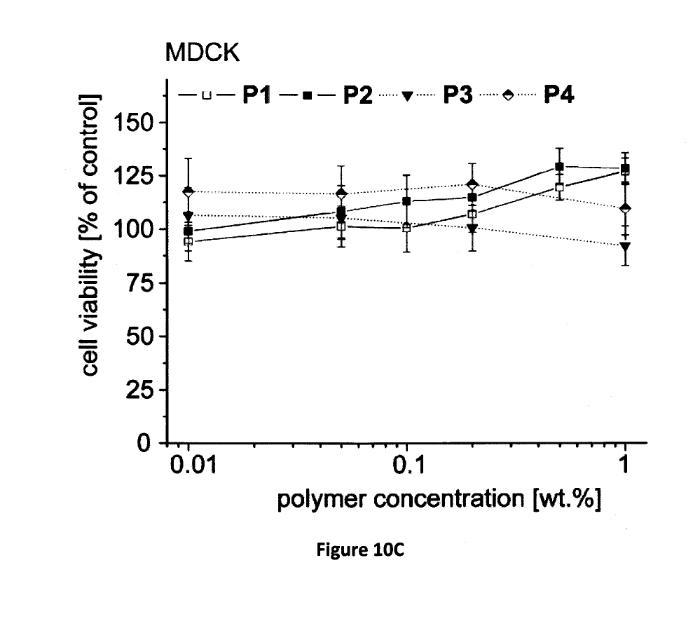

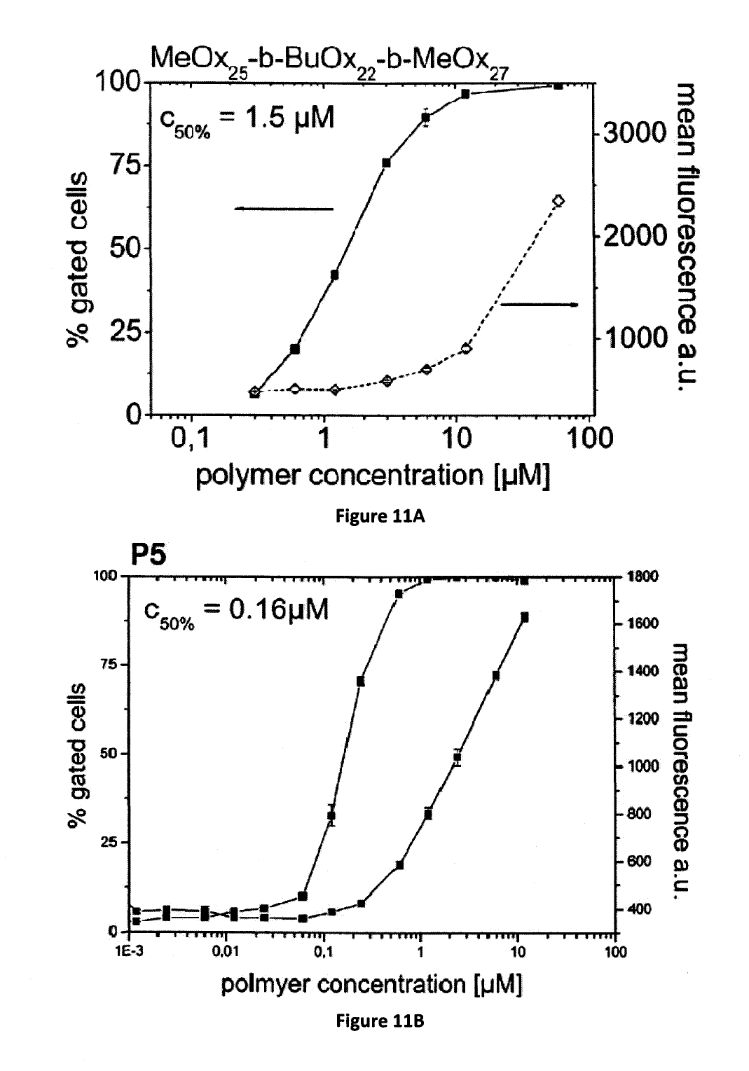

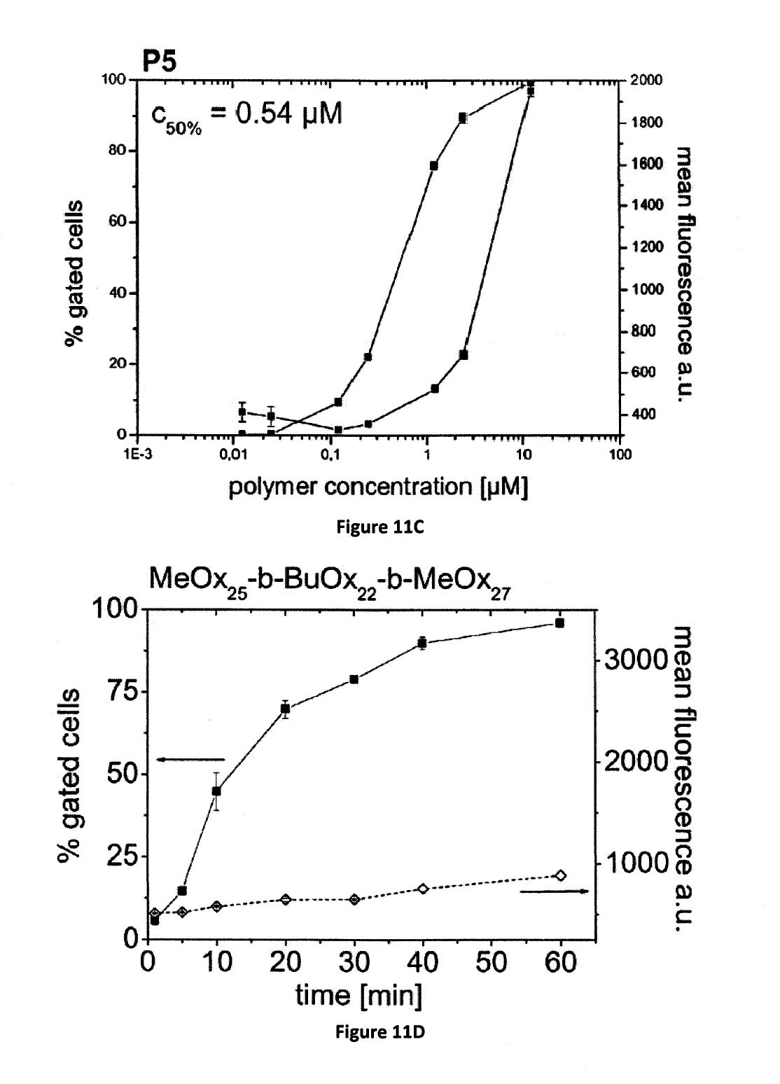

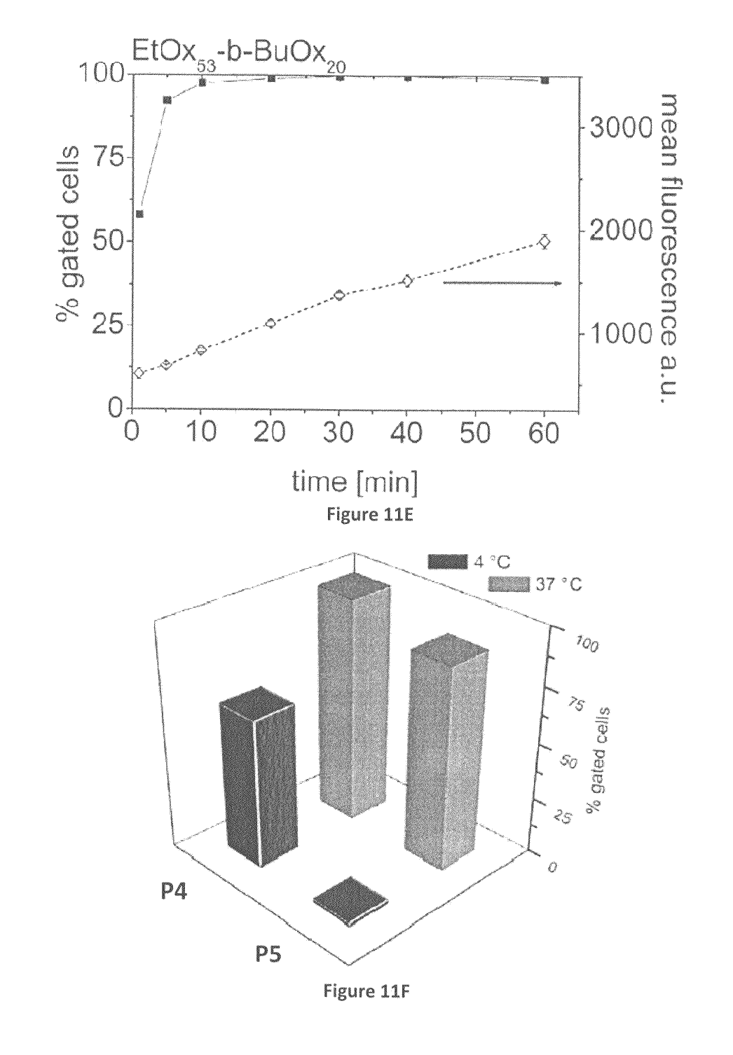

These drug loaded micelles are very small in size (approx 20-50 nm) and show a narrow size distribution as determined by the dynamic light scattering (FIGS. 9A-9D). Such materials are excellently suited for biomedical applications, and in particular systemic administration. P1-P4 alone were not cytotoxic at concentrations of up to 20 mg/mL and 24 hours incubation with different cell lines: MCF7/ADR (human, multidrug resistant) and MCF7 (non-resistant human adenocarcinoma), MDCK (Madin-Darby canine kidney) (FIG. 10) and 3TLL (murine). A fluorescently labeled sample was also prepared. It was as shown that the micelles were readily and rapidly (minutes) taken up into the cells (FIG. 11). For P4 and P5 the cellular uptake was observed even at nanomolar concentrations and followed a typical dose dependent manner. Moreover, the uptake was very fast (within minutes) and temperature dependent, albeit complete inhibition of cellular uptake of P4 was not observed at 4° C.

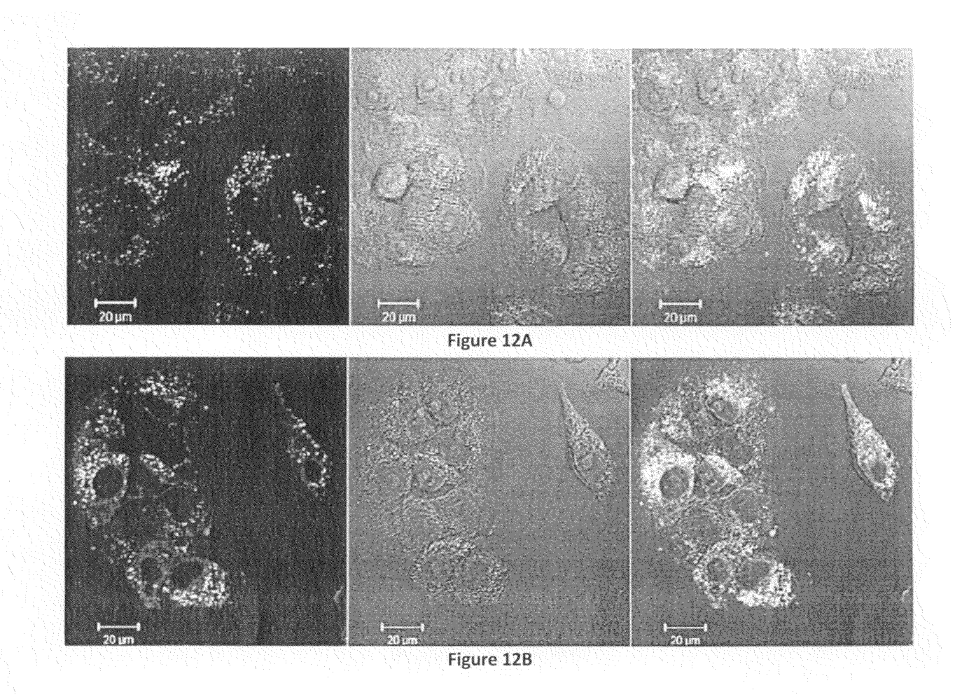

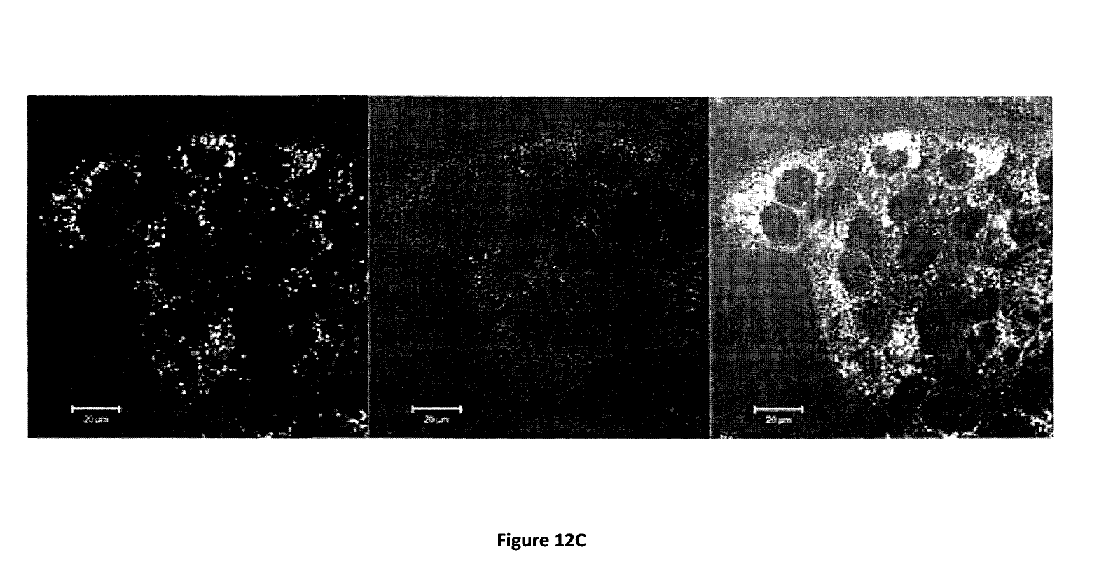

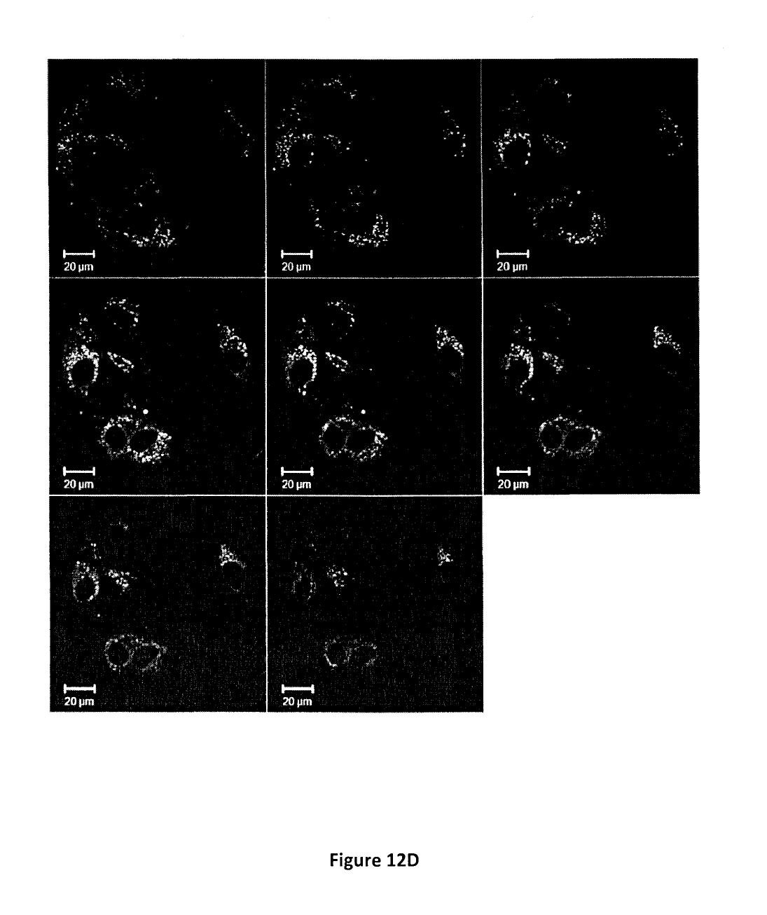

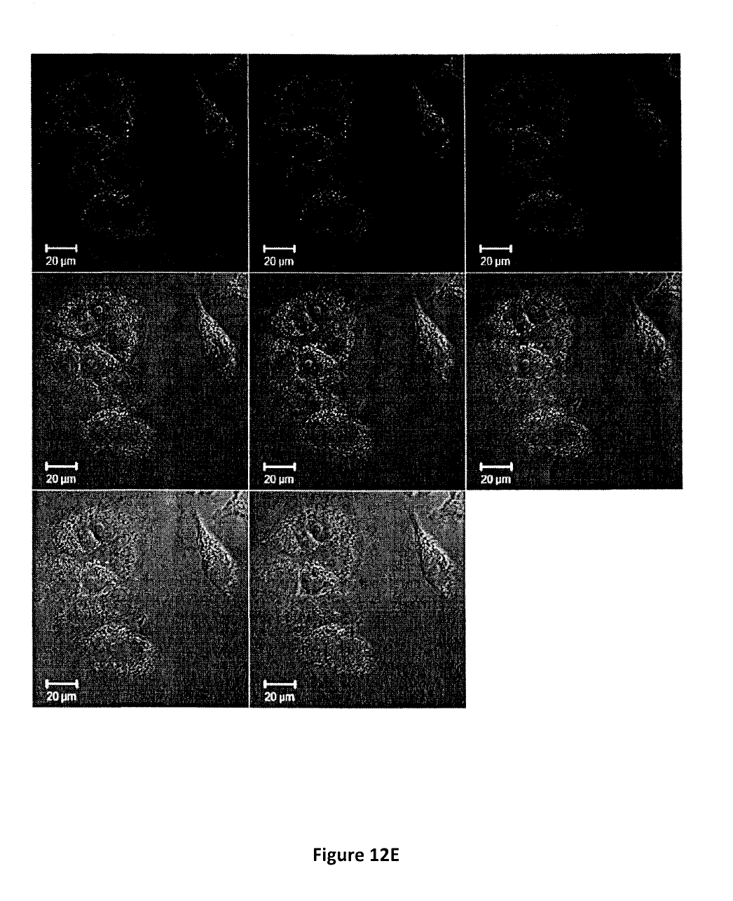

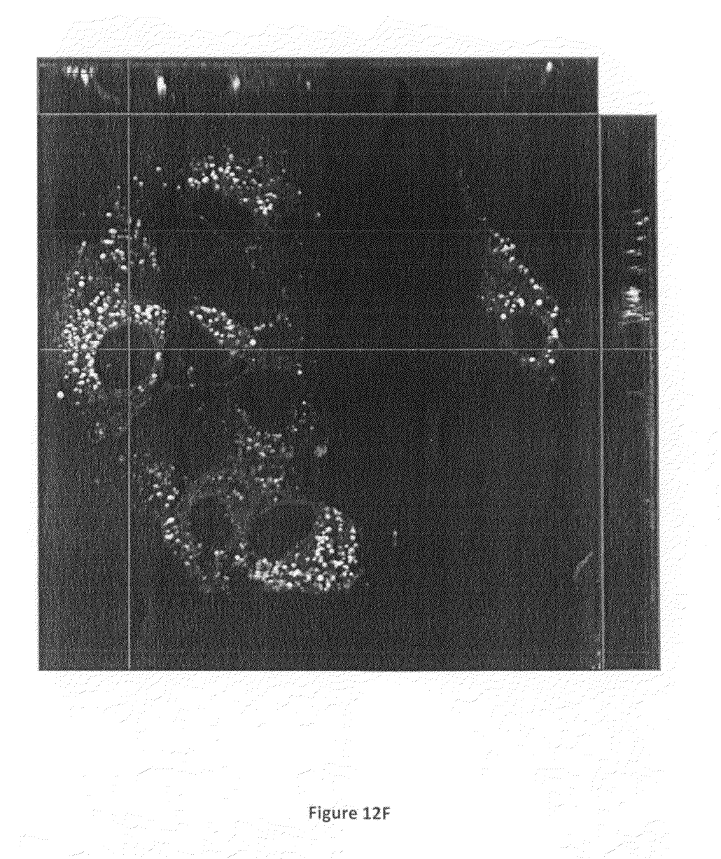

Confocal microscopy confirmed that polymers are internalized. They were found predominantly in small, primarily perinuclear vesicles, although in some cases, e.g. P4, a marked diffuse staining was also observed in the cytosol suggesting that the polymer was not restricted to vesicles (FIG. 12).

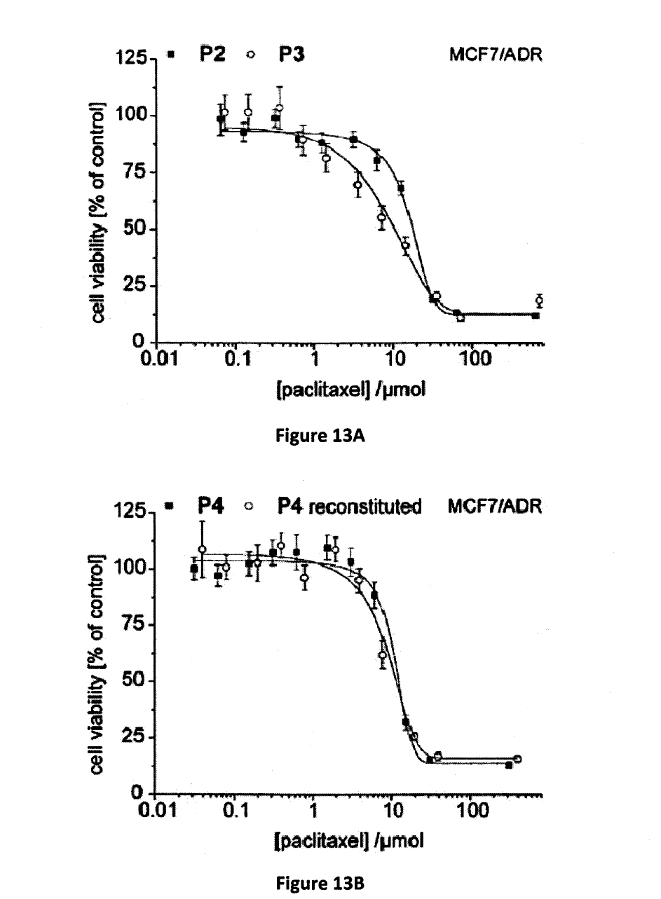

In stark contrast to the plain polymers, the paclitaxel-loaded micelles displayed a pronounced, concentration-dependent toxicity with respect to drug-resistant cells, MCF7/ADR and sensitive cells, MCF7 and 3T-LL. For example, after 24 hour incubation with paclitaxel-loaded P2, P3 and P4, IC₅₀ values in the low micromolar range were observed. Commercially available Taxol® was used as a control and a comparable IC₅₀ was observed. However, in contrast to the poly(2-oxazoline) block copolymers, a Cremophor® EL/ethanol mixture (1/1; v/v) contained in the Taxol® formulation alone (no paclitaxel) has shown considerable toxicity. The paclitaxel-loaded micelles were lyophilized without the need for cryoprotecants and simply be redispersed in water or saline without compromising drug loading, particle size, or in vivo drug efficacy (FIG. 13). The anti-tumor effect of paclitaxel-loaded micelles was examined in C57Bl/6 mice with subcutaneous Lewis Lung carcinoma tumors. Both the poly(2-oxazoline)-based formulation and the regular Taxol® formulation induced significant tumor inhibition on day 15.

The molar masses of these polymers are well below the renal threshold (approx. 65 kDa for globular proteins, 4 nm absolute size) and their polydispersity is reasonably low. Thus, it can be expected that the unimers are readily cleared via the kidney and the drug delivery vehicle can be disposed of appropriately by the organism after it served its purpose.

### Example 10

### Animal Studies

All experiments were performed using female C57/Bl/6 mice 11-12 weeks of age (Taconic Laboratories, Germantown, N.Y.). The animals were kept five per cage with an air filter cover under light (12-hour light/dark cycle) and temperature-controlled (22F1 8C) environment. All manipulations with the animals were performed under a sterilized laminar hood. Food and water were given ad libitum. The animals were treated in accordance to the Principles of Animal Care outlined by National Institutes of Health, and protocols were approved by the Institutional Animal Care and Use Committee of the University of Nebraska Medical Center. Lewis lung carcinoma cells (LLC 3T) were grown in T75 flasks and collected with HBSS. Cell suspensions (1×10⁶ per animal) were injected subcutaneously in a volume of 50 μL on the right flank. After tumors appeared, tumor sizes where recorded (day 1) and treatment solutions were injected at a doses of 10 mg/kg PTX in a volume of 100 μL on day 1, 4 and 7.

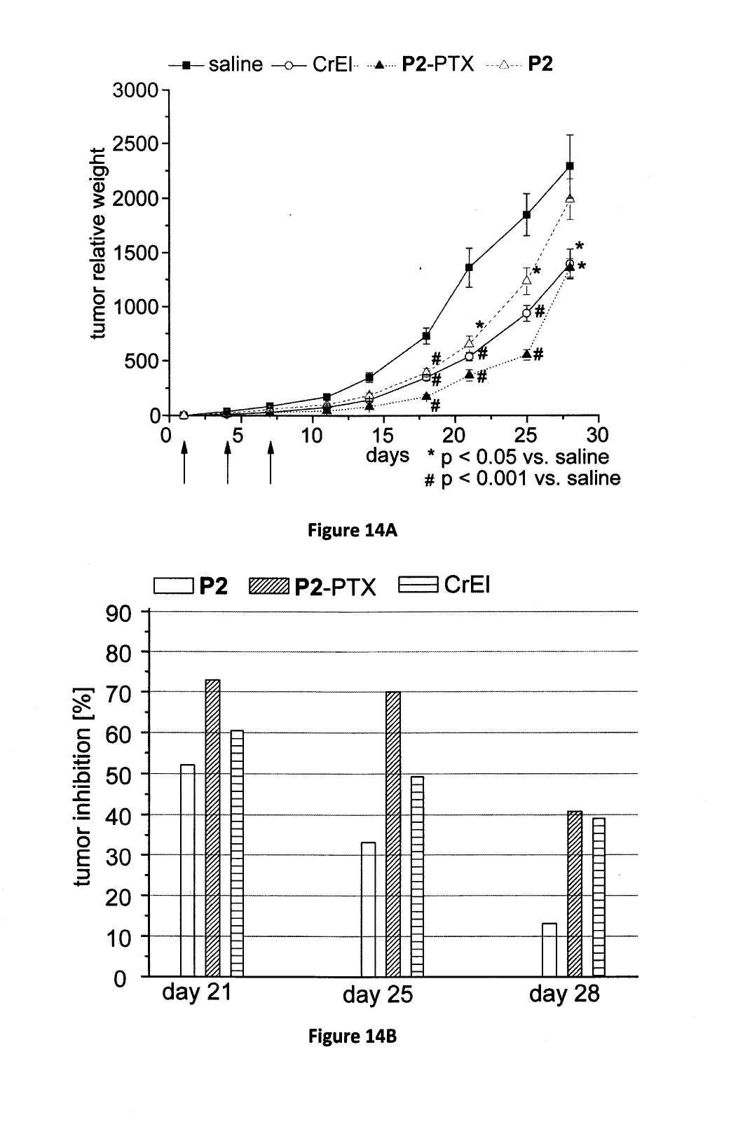

The in vivo anti-tumor effect of PTX-loaded micelles was examined in C57/Bl/6 mice with subcutaneous Lewis Lung carcinoma tumors (FIG. 14). Both commercial (CrEl) and (P2-PTX) formulation significantly (p<0.05) decreased tumor burden after only one injection (day 4, tumor inhibition=72% and 63%, respectively). The tumors in the P2-PTX treated animals remained significantly smaller (p<0.05) than in the animals treated with the commercial product between days 11 and 25. It was found that the tumor inhibition by P2-PTX in this period to be approximately 70% as compared to 50-60% in the CrEl group. After 28 days, however, a sharp increase in the tumor burden of the animals in the P2-PTX regimen was observed and the same tumor inhibition in both treated groups was found.

FIG. 14A shows relative tumor weights of subcutaneous Lewis Lung carcinoma tumors in C57/Bl/6 mice comparing negative controls (saline, P2 alone), treatment with POx solubilized PTX (P2-PTX) and commercial product (CrEl) at the same PTX doses (10 mg/kg). Arrows indicate times of injection. FIG. 14B shows the calculated tumor inhibition in treatment groups of P2, P2-PTX and CrEl at different points of time. Data represented as means±SEM (n=5).

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A composition comprising:
a) at least one amphiphilic block copolymer comprising at least one hydrophilic segment and at least one hydrophobic segment, wherein said hydrophilic segment is a hydrophilic poly(2-oxazoline), and wherein said hydrophobic segment is a hydrophobic poly(2-oxazoline); and
b) at least one hydrophobic compound, wherein said hydrophobic compound has a solubility of less than 1 mg/mL in water or aqueous media at a pH range between 4 and 10.

2. The composition of claim 1 further comprising at least one pharmaceutically acceptable carrier.

3. The composition of claim 1, wherein said hydrophilic segment is poly(2-methyl-2-oxazoline) or poly(2-ethyl-2-oxazoline).

4. The composition of claim 1, wherein said hydrophobic segment has the structure: 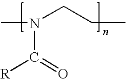
wherein R is an alkyl or an aryl and n is selected between 1 and 300.

5. The composition of claim 4, wherein R comprises 3 to about 50 carbon atoms.

6. The composition of claim 5, wherein R comprises 3 to 6 carbon atoms.

7. The composition of claim 1, wherein said hydrophilic segment is poly(2-butyl-2-oxazoline).

8. The composition of claim 1, wherein said hydrophobic compound is a therapeutic agent.

9. The composition of claim 8, wherein said therapeutic agent is selected from the group consisting of peptides, peptoides, polyenes, macrocyles, glycosides, terpenes, terpenoids, aliphatic compounds, and aromatic compounds.

10. The composition of claim 1, wherein said hydrophobic compound has a solubility of less than 10 μg/mL in water or aqueous media at a pH range between 4 and 10.

11. The composition of claim 1, wherein said amphiphilic block copolymer is selected from the group consisting of a linear block copolymer, a star-like block copolymer, a graft block copolymers, a dendrimer block copolymer, and a hyperbranched block copolymers.

12. The composition of claim 1, wherein said amphiphilic block copolymer is a diblock copolymer or a triblock copolymer consisting of two hydrophilic segments and one hydrophobic segment.

13. The composition of claim 1, wherein said amphiphilic block copolymer and said hydrophobic compound form a soluble aggregate in aqueous media and wherein said aggregate has a size from about 5 nm to about 200 nm.

14. The composition of claim 13, wherein said aggregate has a size from about 10 nm to about 50 nm.

15. The composition of claim 1, wherein said hydrophobic compound and said amphiphilic block copolymer are in a weight ratio of at least 1:10.

16. The composition of claim 1, wherein said hydrophobic compound and said amphiphilic block copolymer are in a weight ratio of at least 4:6.

17. The composition of claim 1, wherein said amphiphilic copolymer comprises the formula: 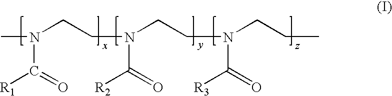
wherein x and y are independently selected between 1 and about 300; z is selected from between 0 and about 300; R₁ and R₃ are independently selected from the group consisting of —H, —OH, —NH₂, —SH, —CH₃, —CH₂CH₃, and an alkyl comprising 1 or 2 carbon atoms; and R₂ is an alkyl or an aryl.

18. The composition of claim 17, wherein R₂ is an alkyl comprising between 3 and 6 carbon atoms.

19. The composition of claim 17, wherein R₁ and R₃ are independently selected from the group consisting of —CH₃ and —CH₂CH₃.

20. A method for delivering at least one hydrophobic compound to a subject, said method comprising administering the composition of claim 1 to said patient.

21. A method of treating a disorder or disease in a patient in need thereof, said method comprising the administration of the composition of claim 8 to said patient.

22. The method of claim 21, wherein said disorder or disease is cancer and the therapeutic agent is a chemotherapeutic agent.

23. The method of claim 22, wherein said chemotherapeutic agent is a taxane.
